(19) United States
(12) Patent Application Publication
(10) Pub. No.: US 2023/0014026 A1
(43) Pub. Date: Jan. 19, 2023

(54) Anti-Tumor Combination Therapy comprising Anti-CD19 Antibody and Polypeptides Blocking the SIRPalpha-CD47 Innate Immune Checkpoint

(71) Applicants: MorphoSys AG, Planegg (DE); Gilead Sciences, Inc., Foster City, CA (US)

(72) Inventors: Jan ENDELL, München (DE); Günter FINGERLE-ROWSON, Starnberg (DE); Mark Ping CHAO, Foster City, CA (US)

(21) Appl. No.: 17/354,442

(22) Filed: Jun. 22, 2021

(30) Foreign Application Priority Data
Jun. 22, 2020 (EP) 20181309.4
Nov. 30, 2020 (EP) 20210588.8

(51) Int. Cl. C07K 16/28; A61P 35/00

(52) U.S. Cl. CPC C07K 16/2803; C07K 16/2896; A61K 2039/507; C07K 2317/76; C07K 2317/565; A61P 35/00

(57) ABSTRACT

The present disclosure is directed to a combination therapy comprising an antibody or antibody fragment specific for CD19 and a polypeptide that blocks the SIRPα-CD47 innate immune checkpoint for use in the treatment of cancer, in particular hematological cancer such as leukemia or lymphoma.

This patent application claims the benefit of priority from EP20210588.8 filed Nov. 30, 2020 and EP20181309.4 filed Jun. 22, 2020, teachings of each of which are incorporated by reference herein in their entireties.

### FIELD OF THE INVENTION

The present disclosure is directed to a combination therapy comprising an anti-CD19 antibody or antibody fragment thereof and polypeptides that block the SIRPα-CD47 innate immune checkpoint for use in the treatment of leukemia or lymphoma.

### BACKGROUND

CD19 is a 95-kDa transmembrane glycoprotein of the immunoglobulin superfamily containing two extracellular immunoglobulin-like domains and an extensive cytoplasmic tail. The protein is a pan-B lymphocyte surface receptor and is ubiquitously expressed from the earliest stages of pre-B cell development onwards until it is down-regulated during terminal differentiation into plasma cells. It is B-lymphocyte lineage specific and not expressed on hematopoietic stem cells and other immune cells, except some follicular dendritic cells. CD19 functions as a positive regulator of B cell receptor (BCR) signalling and is important for B cell activation and proliferation and in the development of humoral immune responses. It acts as a co-stimulatory molecule in conjunction with CD21 and CD81 and is critical for B cell responses to T-cell-dependent antigens. The cytoplasmic tail of CD19 is physically associated with a family of tyrosine kinases that trigger downstream signalling pathways via the src-family of protein tyrosine kinases. CD19 is an attractive target for cancers of lymphoid origin since it is highly expressed in nearly all-chronic lymphocytic leukemia (CLL) and non-Hodgkin's lymphomas (NHL), as well as many other different types of leukemias, including acute lymphocytic leukemia (ALL) and hairy cell leukemia (HCL).

Tafasitamab (former names: MOR208 and XmAb®5574) is a humanized monoclonal antibody that targets the antigen CD19, a transmembrane protein involved in B-cell receptor signalling. Tafasitamab has been engineered in the IgG Fc-region to enhance antibody-dependent cell-mediated cytotoxicity (ADCC), thus improving a key mechanism for tumor cell killing and offering potential for enhanced efficacy compared to conventional antibodies, i.e. non-enhanced antibodies. Tafasitamab has or is currently being studied in several clinical trials, such as in CLL, ALL and NHL. In some of those trials, Tafasitamab is used in combination with Idelalisib, Lenalidomide or Venetoclax.

Despite recent discoveries and developments of several anti-cancer agents, due to poor prognosis for many types of cancers including CD19-expressing tumors, there is still a need for an improved method or therapeutic approach for treating such types of cancers.

Accordingly, the present inventors have confirmed that combined administration of an antibody or antibody fragment specific for CD19 together with a polypeptide that blocks the SIRPα-CD47 innate immune checkpoint has superior effects on the treatment of malignant lymphomas of B cell origin, and have completed the present invention.

### SUMMARY OF INVENTION

The present disclosure provides a novel combination for use in the treatment of cancer, comprising an antibody or antibody fragment specific for CD19 and a polypeptide that blocks the SIRPα-CD47 innate immune checkpoint.

Macrophages are innate immune cells that reside in all tissues. In cancer, macrophages can promote or inhibit tumor growth depending on cellular signals. Characterization of subsets of macrophages has revealed at least 2 subsets; one subset, M2 macrophages, produces arginase and promotes tumor growth while another subset, M1 macrophages, produces nitrous oxide synthetase and mediates tumor killing. Macrophages can kill via antibody dependent mechanisms such as antibody-dependent cellular phagocytosis (ADCP) or antibody independent mechanisms.

Unlike healthy cells, unwanted, aged or dying cells display markers or ligands called “eat-me” signals, i.e. “altered self”, which can in turn be recognized by receptors on phagocytes such as neutrophils, monocytes and macrophages. Healthy cells may display “don't eat-me” signals that actively inhibit phagocytosis; these signals are either downregulated in the dying cells, are present in an altered conformation or they are superseded by the upregulation of “eat me” or pro-phagocytic signals. The cell surface protein CD47 on healthy cells and its engagement of a phagocyte receptor, Signal Regulatory Protein a (SIRPα), constitutes a key “don't eat-me” signal that can turn off engulfment mediated by multiple modalities, including apoptotic cell clearance and FcR mediated phagocytosis. Blocking the CD47 mediated engagement of SIRP on a phagocyte, or the loss of CD47 expression in knockout mice, can cause removal of live cells and non-aged erythrocytes. Blocking SIRPoc also allows engulfment of targets that are not normally phagocytosed, for those cells where pre-phagocytic signals are also present.

CD47 is a broadly expressed transmembrane glycoprotein with a single Ig-like domain and five membrane spanning regions, which functions as a cellular ligand for SIRPoc, with binding mediated through the NH2-terminal V-like domain of SIRPoc. SIRPoc is expressed primarily on myeloid cells, including macrophages, granulocytes, myeloid dendritic cells (DCs), mast cells, and their precursors, including hematopoietic stem cells. Structural determinants on SIRPoc that mediate CD47 binding are discussed by Lee et al. (2007) J. Immunol. 179:7741-7750; Hatherley et al. (2007) J. B. C. 282:14567-75; and the role of SIRPoc cis dimerization in CD47 binding is discussed by Lee et al. (2010) J. B. C. 285:37953-63. In keeping with the role of CD47 to inhibit phagocytosis of normal cells, there is evidence that it is transiently upregulated on hematopoietic stem cells (HSCs) and progenitors just prior to and during their migratory phase, and that the level of CD47 on these cells determines the probability that they are engulfed in vivo.

CD47 is overexpressed in all cancers tested to date. Indeed it has been shown that CD47 is over-expressed on tumor versus normal cells by approximately 3.3 fold (Majeti et al (2009) Cell 138:286-289: Willingham et al (2012) PNAS 109:6662-6667).

Programmed cell death (PCD) and phagocytic cell removal are amongst the ways that an organism responds in order to remove damaged, precancerous, or infected cells. Thus, the cells that survive this organismal response (e.g., cancerous cells, chronically infected cells, etc.) have devised ways to evade PCD and phagocytic cell removal. CD47, the “don't eat me” signal, is constitutively upregulated on a wide variety of diseased cells, cancer cells, and infected cells, allowing these cells to evade phagocytosis. Anti-CD47 agents that block the interaction between CD47 on one cell (e.g., a cancer cell, an infected cell, etc.) and SIRPoc on another cell (e.g., a phagocytic cell) counteract the increase of CD47 expression and facilitate the phagocytosis of the cancer cell and/or the infected cell. Thus, anti-CD47 agents can be used to treat and/or protect against a wide variety of conditions/disorders.

In the present disclosure the inventors have combined the CD19-targeting antibody Tafasitamab (Fc-enhanced) and a CD47-targeting antibody and evaluated the anti-tumor activity. In vitro and in vivo, a significant increased anti-tumor effect was observed when Tafasitamab was combined with a CD47-targeting antibody.

In summary, it is demonstrated that administration of Tafasitamab and blockade of the SIRPα-CD47 innate immune checkpoint, e.g. via CD47-targeting antibody or SIRPα-targeting antibody may hold a promising approach for lymphoma and leukemia therapy.

Provided herein is pharmaceutical combination comprising an anti-CD19 antibody or antibody fragment thereof and a polypeptide that blocks the SIRPα-CD47 innate immune checkpoint for use in the treatment of hematological cancer. In some embodiments the hematological cancer is chronic lymphocytic leukemia (CLL), non-Hodgkin's lymphoma (NHL), small lymphocytic lymphoma (SLL) or acute lymphoblastic leukemia (ALL).

In one aspect the present disclosure provides a pharmaceutical combination comprising an anti-CD19 antibody or antibody fragment thereof and a polypeptide that blocks the SIRPα-CD47 innate immune checkpoint for use in the treatment of hematological cancer, wherein said polypeptide that blocks the SIRPα-CD47 innate immune checkpoint is an antibody or antibody fragment that specifically binds to human CD47 or human SIRPα or a polypeptidic SIRPα reagent.

In another aspect the present disclosure provides a kit comprising an anti-CD19 antibody or antibody fragment thereof and instructions to administer said anti-CD19 antibody or antibody fragment thereof in combination with a polypeptide that blocks the SIRPα-CD47 innate immune checkpoint. In an embodiment said polypeptide that blocks the SIRPα-CD47 innate immune checkpoint is an antibody or antibody fragment that specifically binds to human CD47 or human SIRPα or a polypeptidic SIRPα reagent.

In one aspect the present disclosure provides a pharmaceutical combination, comprising an antibody or antibody fragment specific for CD19 and a polypeptide that blocks the SIRPα-CD47 innate immune checkpoint for use in the treatment of cancer wherein the antibody or antibody fragment specific for CD19 comprises a heavy chain variable region comprising an HCDR1 region comprising the sequence SYVMH (SEQ ID NO: 1), an HCDR2 region comprising the sequence NPYNDG (SEQ ID NO: 2), and an HCDR3 region comprising the sequence GTYYYGTRVFDY (SEQ ID NO: 3) and a light chain variable region comprising comprising the sequence LCDR1 region comprising the sequence RSSKSLQNVNGNTYLY (SEQ ID NO: 4), an LCDR2 region comprising the sequence RMSNLNS (SEQ ID NO: 5), and an LCDR3 region comprising the sequence MQHLEYPIT (SEQ ID NO: 6) for use in the treatment of cancer.

In one aspect the present disclosure provides a pharmaceutical combination, comprising an antibody or antibody fragment specific for CD19 and a polypeptide that blocks the SIRPα-CD47 innate immune checkpoint wherein said antibody or antibody fragment specific for CD19 comprises a heavy chain variable region comprising an HCDR1 region of SYVMH (SEQ ID NO: 1), an HCDR2 region of NPYNDG (SEQ ID NO: 2), and an HCDR3 region of GTYYYGTRVFDY. (SEQ ID NO: 3) and a light chain variable region comprising an LCDR1 region of RSSKSLQNVNGNTYLY (SEQ ID NO: 4), an LCDR2 region of RMSNLNS (SEQ ID NO: 5), and an LCDR3 region of MQHLEYPIT (SEQ ID NO: 6) for use in the treatment of cancer.

In another aspect the antibody or antibody fragment specific for CD19 comprises a heavy chain variable region of

(SEQ ID NO: 7)
EVQLVESGGGLVKPGGSLKLSCAASGYTFTSYVMHWVRQAPGKGLEWIGY
INPYNDGTKYNEKFQGRVTISSDKSISTAYMELSSLRSEDTAMYYCARGT
YYYGTRVFDYWGQGTLVTVSS

and a light chain variable region of

(SEQ ID NO: 8)
DIVMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIK.

In another aspect the antibody or antibody fragment specific for CD19 has effector function. In another aspect the antibody or antibody fragment specific for CD19 has an enhanced effector function. In one embodiment the effector function is ADCC. In one embodiment the antibody or antibody fragment specific for CD19 has an enhanced ADCC activity. In a further embodiment the antibody or antibody fragment specific for CD19 comprises an Fc domain comprising an amino acid substitution at position S239 and/or I332, wherein the numbering is according to the EU index as in Kabat.

In yet another aspect the antibody or antibody fragment specific for CD19 comprises a heavy chain constant region of

(SEQ ID NO: 9)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPDVFLFPPKPKDTLMISRTPEVTCVVVDVS
HEDPEVQFNWYVDGVEVHNAKTKPREEQFNSTFRVVSVLTVVHQDWLNGK
EYKCKVSNKALPAPEEKTISKTKGQPREPQVYTLPPSREEMTKNQVSLTC
LVKGFYPSDIAVEWESNGQPENNYKTTPPMLDSDGSFFLYSKLTVDKSRW
QQGNVFSCSVMHEALHNHYTQKSLSLSPGK.

In a further aspect the antibody specific for CD19 comprises a light chain constant region of

(SEQ ID NO: 10)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC.

In yet another aspect the antibody specific for CD19 comprises a heavy chain constant region of

(SEQ ID NO: 9)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPDVFLFPPKPKDTLMISRTPEVTCVVVDVS
HEDPEVQFNWYVDGVEVHNAKTKPREEQFNSTFRVVSVLTVVHQDWLNGK
EYKCKVSNKALPAPEEKTISKTKGQPREPQVYTLPPSREEMTKNQVSLTC
LVKGFYPSDIAVEWESNGQPENNYKTTPPMLDSDGSFFLYSKLTVDKSRW
QQGNVFSCSVMHEALHNHYTQKSLSLSPGK

and a light chain constant region of

(SEQ ID NO: 10)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC.

In yet another aspect the antibody specific for CD19 comprises a heavy chain region of

(SEQ ID NO: 11)
EVQLVESGGGLVKPGGSLKLSCAASGYTFTSYVMHWVRQAPGKGLEWIGY
INPYNDGTKYNEKFQGRVTISSDKSISTAYMELSSLRSEDTAMYYCARGT
YYYGTRVFDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPDVFLFPPK
PKDTLMISRTPEVTCVVVDVSHEDPEVQFNWYVDGVEVHNAKTKPREEQF
NSTFRVVSVLTVVHQDWLNGKEYKCKVSNKALPAPEEKTISKTKGQPREP
QVYTLPPSREEMTKNQVSLTCLVKGFYPSDIAVEWESNGQPENNYKTTPP
MLDSDGSFFLYSKLTVDKSRWQQGNVFSCSVMHEALHNHYTQKSLSLSPG
K

and a light chain region of

(SEQ ID NO: 12)
DIVMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC.

In one aspect the present disclosure provides a pharmaceutical combination comprising an anti-CD19 antibody or antibody fragment thereof and a polypeptide that blocks the SIRPα-CD47 innate immune checkpoint for use in the treatment of cancer wherein the cancer is a hematological cancer. In one embodiment the hematological cancer is chronic lymphocytic leukemia (CLL), non-Hodgkin's lymphoma (NHL), small lymphocytic lymphoma (SLL) or acute lymphoblastic leukemia (ALL). In another embodiment the hematological cancer is non-Hodgkin's lymphoma (NHL). In a further embodiment the non-Hodgkin's lymphoma is selected from the group consisting of follicular lymphoma, small lymphocytic lymphoma, mucosa-associated lymphoid tissue, marginal zone lymphoma, diffuse large B cell lymphoma, Burkitt's lymphoma and mantle cell lymphoma.

In one aspect the present disclosure provides pharmaceutical combination comprising an anti-CD19 antibody or antibody fragment thereof and a polypeptide that blocks the SIRPα-CD47 innate immune checkpoint for use in the treatment of cancer, wherein the antibody specific for CD19 and the polypeptide that blocks the SIRPα-CD47 innate immune checkpoint are administered in a separate manner.

In one aspect the present disclosure provides pharmaceutical combination comprising an anti-CD19 antibody or antibody fragment thereof and a polypeptide that blocks the SIRPα-CD47 innate immune checkpoint for use in the treatment of cancer, wherein the antibody specific for CD19 and the polypeptide that blocks the SIRPα-CD47 innate immune checkpoint are administered in a simultaneous manner.

In one aspect the present disclosure provides a pharmaceutical combination comprising an anti-CD19 antibody or antibody fragment thereof and an anti-CD47 antibody or antibody fragment thereof for use in the treatment of hematological cancer, wherein the anti-CD19 antibody or antibody fragment thereof comprises a heavy chain variable region of

(SEQ ID NO: 7)
EVQLVESGGGLVKPGGSLKLSCAASGYTFTSYVMHWVRQAPGKGLEWIGY
INPYNDGTKYNEKFQGRVTISSDKSISTAYMELSSLRSEDTAMYYCARGT
YYYGTRVFDYWGQGTLVTVSS
and

a light chain variable region of

(SEQ ID NO: 8)
DIVMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIK

and wherein the anti-CD47 antibody or fragment thereof comprises a heavy chain variable region of

(SEQ ID NO: 30)
QVQLVQSGAEVKKPGASVKVSCKASGYTFTNYNMHWVRQAPGQRLEWMGT
IYPGNDDTSYNQKFKDRVTITADTSASTAYMELSSLRSEDTAVYYCARGG
YRAMDYWGQGTLVTVSS

and a light chain variable region of

DIVMTQSPLSLPVTPGEPASISCRSSQSIVYSNGNTYLGWYLQKPGQSPQLLIYKVS NRFSGVPDRFSGSGSGTDFTLKISRVEAEDVGVYYCFQGSHVPYTFGQGTKLEIK (SEQ ID NO:31). In one embodiment the hematological cancer is chronic lymphocytic leukemia (CLL), non-Hodgkin's lymphoma (NHL), small lymphocytic lymphoma (SLL) or acute lymphoblastic leukemia (ALL). In another embodiment the hematological cancer is non-Hodgkin's lymphoma (NHL). In a further embodiment the non-Hodgkin's lymphoma is selected from the group consisting of follicular lymphoma, small lymphocytic lymphoma, mucosa-associated lymphoid tissue, marginal zone lymphoma, diffuse large B cell lymphoma, Burkitt's lymphoma and mantle cell lymphoma. In another embodiment the hematological cancer is diffuse large B cell lymphoma.

In one aspect the present disclosure provides a pharmaceutical combination comprising an anti-CD19 antibody or antibody fragment thereof and an anti-CD47 antibody or antibody fragment thereof for use in the treatment of hematological cancer, wherein the anti-CD19 antibody or antibody fragment thereof comprises a heavy chain region of

(SEQ ID NO: 11)
EVQLVESGGGLVKPGGSLKLSCAASGYTFTSYVMHWVRQAPGKGLEWIGY
INPYNDGTKYNEKFQGRVTISSDKSISTAYMELSSLRSEDTAMYYCARGT
YYYGTRVFDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPDVFLFPPK
PKDTLMISRTPEVTCVVVDVSHEDPEVQFNWYVDGVEVHNAKTKPREEQF
NSTFRVVSVLTVVHQDWLNGKEYKCKVSNKALPAPEEKTISKTKGQPREP
QVYTLPPSREEMTKNQVSLTCLVKGFYPSDIAVEWESNGQPENNYKTTPP
MLDSDGSFFLYSKLTVDKSRWQQGNVFSCSVMHEALHNHYTQKSLSLSPG
K

and a light chain region of

(SEQ ID NO: 12)
DIVMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC

and wherein the anti-CD47 antibody or fragment thereof comprises a heavy chain of

(SEQ ID NO: 34)
QVQLVQSGAEVKKPGASVKVSCKASGYTFTNYNMHWVRQAPGQRLEWMGT
IYPGNDDTSYNQKFKDRVTITADTSASTAYMELSSLRSEDTAVYYCARGG
YRAMDYWGQGTLVTVSSASTKGPSVFPLAPCSRSTSESTAALGCLVKDYF
PEPVTVSWNSGALTSGVHTFPAVLQSSGLYSLSSVVTVPSSSLGTKTYTC
NVDHKPSNTKVDKRVESKYGPPCPPCPAPEFLGGPSVFLFPPKPKDTLMI
SRTPEVTCVVVDVSQEDPEVQFNWYVDGVEVHNAKTKPREEQFNSTYRVV
SVLTVLHQDWLNGKEYKCKVSNKGLPSSIEKTISKAKGQPREPQVYTLPP
SQEEMTKNQVSLTCLVKGFYPSDIAVEWESNGQPENNYKTTPPVLDSDGS
FFLYSRLTVDKSRWQEGNVFSCSVMHEALHNHYTQKSLSLSLGK

and a light chain of

(SEQ ID NO: 35)
DIVMTQSPLSLPVTPGEPASISCRSSQSIVYSNGNTYLGWYLQKPGQSPQ
LLIYKVSNRFSGVPDRFSGSGSGTDFTLKISRVEAEDVGVYYCFQGSHVP
YTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVIKSFNRGEC.

In one embodiment the hematological cancer is chronic lymphocytic leukemia (CLL), non-Hodgkin's lymphoma (NHL), small lymphocytic lymphoma (SLL) or acute lymphoblastic leukemia (ALL). In another embodiment the hematological cancer is non-Hodgkin's lymphoma (NHL). In a further embodiment the non-Hodgkin's lymphoma is selected from the group consisting of follicular lymphoma, small lymphocytic lymphoma, mucosa-associated lymphoid tissue, marginal zone lymphoma, diffuse large B cell lymphoma, Burkitt's lymphoma and mantle cell lymphoma. In another embodiment the hematological cancer is diffuse large B cell lymphoma.

### BRIEF DESCRIPTION OF THE DRAWINGS

FIG. 1: Antigen expression levels of relevant surface antigens on cell lines used for ADCP assays. 1.0E+05 Raji, Ramos, Daudi or SU-DHL-6 cells were plated, blocked for 30 minutes with 50 μg/mL of human gamma globulin and stained with commercial primary labeled antibodies or suitable isotype controls for 30 minutes. Assay readout was done with NovoCyte or NovoCyte Quanteon instrument and data were analyzed with NovoCyte software. Data are shown in bar-graphs showing MFI (mean fluorescence intensity) ratio values, calculated with normalization of MFI value of the antigen of interest on MFI value of suitable isotype control.

FIG. 2, A and B and FIG. 3, C and D: Addition of anti-CD47 mAb increases tafasitamab mediated ADCP. CFSE stained THP-1 cells were used as effector cells and co-incubated with Cell Trace™ Violet stained Raji (A), Ramos (B), Daudi (C) or SU-DHL-6 (D) target cells in E:T ratio 2:1 for 4 hours at 37° C. and 5% CO2. Gating on target cells as 100% was used for ADCP analysis. Effector cell-mediated unspecific phagocytosis of tumor cells was determined by incubation of effector cells with target cells in the absence of the antibody and is shown on graphs as dotted gray line named background ADCP. A flow cytometry-based readout was utilized to measure the phagocytosis of target cells by quantifying the effector cells which phagocytosed target cells and are therefore double positive for both used staining dyes. Percent of phagocytosis represents the percentage of double positive cells, when total target cells correspond to 100 percent. Black dotted curves stand for tafasitamab titration, while tafasitamab titration with addition of 3 nM anti-CD47 antibody (clone B6H12.2) is shown with full black lines. Error bars represents standard deviation of technical replicates. Gray dotted line indicates background phagocytosis without addition of an antibody.

FIG. 4, A and B: Addition of anti-CD47 mAb increases tafasitamab mediated ADCP. CFSE stained THP-1 cells were used as effector cells and co-incubated with Cell Trace™ Violet stained Raji (A) or Ramos (B) target cells in E:T ratio 2:1 for 4 hours at 37° C. and 5% CO2. Gating on target cells as 100% was used for ADCP analysis. Effector cell-mediated unspecific phagocytosis of tumor cells was determined by incubation of effector cells with target cells in the absence of the antibody and is shown on graphs as dotted gray line named background ADCP. A flow cytometry-based readout was utilized to measure the phagocytosis of target cells by quantifying the effector cells which phagocytosed target cells and are therefore double positive for both used staining dyes. Percent of phagocytosis represents the percentage of double positive cells, when total target cells correspond to 100 percent. Black dotted curves stand for tafasitamab titration, while tafasitamab titration with addition of 3 nM anti-CD47 antibody (clone B6H12.2) is shown with full black lines. Gray dotted line indicates background phagocytosis without addition of an antibody.

FIG. 5: Efficacy of MOR208 & anti-CD47 antibody combinations in an disseminated survival model (MOR208P014)

FIG. 6: MOR208 combinational efficacy in Ramos-SCID subcutaneous tumors (MOR208P015)

FIG. 7: MOR208 combinational efficacy in Ramos-NOD-SCID subcutaneous tumors (MOR208P016)

FIG. 8: Efficacy of MOR208 & CD47/SIRPα checkpoint increases phagocytosis of Ramos cells in ADCP assays with M1 and M2 macrophages used as effector cells.

FIG. 9, A, B, C and D: Co-treatment with magrolimab plus tafasitamab enhances phagocytosis of different lymphoma cells. Fluorescent labeled Raji cells (A), Toledo cells (B), U2932 cells (C) or RCK8 cells (D) were co-incubated with ex vivo differentiated human macrophages at a ratio of 2:1, along with the indicated antibody treatments at a concentration of 10 μg/mL, for 2 hours at 37° C. Macrophages were identified by staining with an antibody against cell surface marker CD11b, and the reactions were assessed by flow cytometry. Phagocytic events were defined as the percent of total macrophages also positive for the tumor cell-specific fluorescent signal, corresponding to macrophages which had engulfed tumor cells. Phagocytosis of lymphoma cells was increased by treatment with either magrolimab or tafasitamab; and this phagocytosis was enhanced by the combination of the two drugs.

FIG. 10, A and B: Magrolimab and tafasitamab enhance phagocytosis of CA46 lymphoma cells, but do not show a combinatorial effect. Fluorescent labeled CA46 (A) or JVM-2 cells (B) cells were co-incubated with ex vivo differentiated human macrophages at a ratio of 2:1, along with the indicated antibody treatments at a concentration of 10 μg/mL, for 2 hours at 37° C. Macrophages were identified by staining with an antibody against cell surface marker CD11b, and the reactions were assessed by flow cytometry. Phagocytic events were defined as the percent of total macrophages also positive for the tumor cell-specific fluorescent signal, corresponding to macrophages which had engulfed tumor cells. Phagocytosis of CA46 cells and JVM-2 cells was increased by treatment with either magrolimab or tafasitamab; but this phagocytosis was not clearly enhanced by the combination of the two drugs.

### DEFINITIONS

The term “CD19” refers to the protein known as CD19, having the following synonyms: B4, B-lymphocyte antigen CD19, B-lymphocyte surface antigen B4, CVID3, Differentiation antigen CD19, MGC12802, and T-cell surface antigen Leu-12.

Human CD19 has the amino acid sequence of:

(SEQ ID NO: 13)
MPPPRLLFFLLFLTPMEVRPEEPLVVKVEEGDNAVLQCLKGTSDGPTQQL
TWSRESPLKPFLKLSLGLPGLGIHMRPLAIWLFIFNVSQQMGGFYLCQPG
PPSEKAWQPGWTVNVEGSGELFRWNVSDLGGLGCGLKNRSSEGPSSPSGK
LMSPKLYVWAKDRPEIWEGEPPCLPPRDSLNQSLSQDLTMAPGSTLWLSC
GVPPDSVSRGPLSWTHVHPKGPKSLLSLELKDDRPARDMWVMETGLLLPR
ATAQDAGKYYCHRGNLTMSFHLEITARPVLWHWLLRTGGWKVSAVTLAYL
IFCLCSLVGILHLQRALVLRRKRKRMTDPTRRFFKVTPPPGSGPQNQYGN
VLSLPTPTSGLGRAQRWAAGLGGTAPSYGNPSSDVQADGALGSRSPPGVG
PEEEEGEGYEEPDSEEDSEFYENDSNLGQDQLSQDGSGYENPEDEPLGPE
DEDSFSNAESYENEDEELTQPVARTMDFLSPHGSAWDPSREATSLGSQSY
EDMRGILYAAPQLRSIRGQPGPNHEEDADSYENMDNPDGPDPAWGGGGRM
GTWSTR

“MOR208” and “XmAb 5574” and “tafasitamab” are used as synonyms for the anti-CD19 antibody according to Table 1. Table 1 provides the amino acid sequences of MOR208/tafasitamab. The MOR208 antibody is described in U.S. patent application Ser. No. 12/377,251, which is incorporated by reference in its entirety. U.S. patent application Ser. No. 12/377,251 describes the antibody named 4G7 H1.52 Hybrid S239D/I332E/4G7 L1.155 (later named MOR208 and tafasitamab).

The term “antibody” as used herein refers to a protein comprising at least two heavy (H) chains and two light (L) chains inter-connected by disulfide bonds, which interacts with an antigen. Each heavy chain is comprised of a variable heavy chain region (abbreviated herein as VH) and a heavy chain constant region. The heavy chain constant region is comprised of three domains, CH1, CH2 and CH3. Each light chain is comprised of a variable light chain region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FR's arranged from amino-terminus to carboxy-terminus in the following order: FR1, CDR1, FR2, CDR2, FR3, CDR3, and FR4. The variable regions of the heavy and light chains contain a binding domain that interacts with an antigen. The term “antibody” includes for example, monoclonal antibodies, human antibodies, humanized antibodies, camelised antibodies and chimeric antibodies. The antibodies can be of any isotype (e.g., IgG, IgE, IgM, IgD, IgA and IgY), class (e.g., IgG1, IgG2, IgG3, IgG4, IgA1 and IgA2) or subclass. Both the light and heavy chains are divided into regions of structural and functional homology.

The phrase “antibody fragment”, as used herein, refers to one or more portions of an antibody that retain the ability to specifically interact with (e.g., by binding, steric hindrance, stabilizing spatial distribution) an antigen. Examples of binding fragments include, but are not limited to, a Fab fragment, a monovalent fragment consisting of the VL, VH, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., (1989) Nature 341:544-546), which consists of a VH domain; and an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al., (1988) Science 242:423-426; and Huston et al., (1988) Proc. Natl. Acad. Sci. 85:5879-5883).. Such single chain antibodies are also intended to be encompassed within the term “antibody fragment”. These antibody fragments are obtained using conventional techniques known to those of skill in the art, and the fragments are screened for utility in the same manner as are intact antibodies. Antibody fragments can also be incorporated into single domain antibodies, maxibodies, minibodies, intrabodies, diabodies, triabodies, tetrabodies, v-NAR and bis-scFv (see, e.g., Hollinger and Hudson, (2005) Nature Biotechnology 23:1126-1136). Antibody fragments can be grafted into scaffolds based on polypeptides such as Fibronectin type III (Fn3) (see U.S. Pat. No. 6,703,199, which describes fibronectin polypeptide monobodies). Antibody fragments can be incorporated into single chain molecules comprising a pair of tandem Fv segments (VH-CH1-VH-CH1) which, together with complementary light chain polypeptides, form a pair of antigen-binding sites (Zapata et al., (1995) Protein Eng. 8:1057-1062; and U.S. Pat. No. 5,641,870).

“Administered” or “administration” includes but is not limited to delivery of a drug by an injectable form, such as, for example, an intravenous, intramuscular, intradermal or subcutaneous route or mucosal route, for example, as a nasal spray or aerosol for inhalation or as an ingestible solution, capsule or tablet. Preferably, the administration is by an injectable form.

The term “effector function” refers to those biological activities attributable to the Fc region of an antibody, which vary with the antibody isotype. Non-limiting examples of antibody effector functions include C1q binding and complement dependent cytotoxicity (CDC); Fc receptor binding and antibody-dependent cell-mediated cytotoxicity (ADCC) and/or antibody-dependent cellular phagocytosis (ADCP); down regulation of cell surface receptors (e.g. B cell receptor); and B cell activation.

“Antibody-dependent cell-mediated cytotoxicity” or “ADCC” refers to a form of cytotoxicity in which antibodies bound onto Fc receptors (FcRs) present on certain cytotoxic cells (e.g. NK cells, neutrophils, and macrophages) enable these cytotoxic effector cells to bind specifically to an antigen-bearing target cell and subsequently kill the target cell with cytotoxins. The primary cells for mediating ADCC, NK cells, express FcyRIII only, whereas monocytes express FcyRI, FcyRII, and FcyRIII.

“Complement-dependent cytotoxicity” or “CDC” refers to the lysis of a target cell in the presence of complement. Activation of the classical complement pathway is initiated by the binding of the first component of the complement system (C1q) to antibodies (of the appropriate subclass) of the present disclosure, which are bound to their cognate antigen.

“Antibody-dependent cellular phagocytosis” or “ADCP” refers to a mechanism of elimination of antibody-coated target cells by internalization by phagocytic cells, such as macrophages or dendritic cells.

The term “hematologic cancer” includes blood-borne tumors and diseases or disorders involving abnormal cell growth and/or proliferation in tissues of hematopoietic origin, such as lymphomas, leukemias, and myelomas.

Non-Hodgkin's lymphoma (“NHL”) is a heterogeneous malignancy originating from lymphocytes. In the United States (U.S.), the incidence is estimated at 65,000/year with mortality of approximately 20,000 (American Cancer Society, 2006; and SEER Cancer Statistics Review). The disease can occur in all ages, the usual onset begins in adults over 40 years, with the incidence increasing with age. NHL is characterized by a clonal proliferation of lymphocytes that accumulate in the lymph nodes, blood, bone marrow and spleen, although any major organ may be involved. The current classification system used by pathologists and clinicians is the World Health Organization (WHO) Classification of Tumours, which organizes NHL into precursor and mature B-cell or T-cell neoplasms. The PDQ is currently dividing NHL as indolent or aggressive for entry into clinical trials. The indolent NHL group is comprised primarily of follicular subtypes, small lymphocytic lymphoma, MALT (mucosa-associated lymphoid tissue), and marginal zone; indolent encompasses approximately 50% of newly diagnosed B-cell NHL patients. Aggressive NHL includes patients with histologic diagnoses of primarily diffuse large B cell (DLBL, “DLBCL”, or DLCL) (40% of all newly diagnosed patients have diffuse large cell), Burkitt's, and mantle cell (“MCL”). The clinical course of NHL is highly variable. A major determinant of clinical course is the histologic subtype. Most indolent types of NHL are considered to be incurable disease. Patients respond initially to either chemotherapy or antibody therapy and most will relapse. Studies to date have not demonstrated an improvement in survival with early intervention. In asymptomatic patients, it is acceptable to “watch and wait” until the patient becomes symptomatic or the disease pace appears to be accelerating. Over time, the disease may transform to a more aggressive histology. The median survival is 8 to 10 years, and indolent patients often receive 3 or more treatments during the treatment phase of their disease. Initial treatment of the symptomatic indolent NHL patient historically has been combination chemotherapy. The most commonly used agents include: cyclophosphamide, vincristine and prednisone (CVP); or cyclophosphamide, adriamycin, vincristine, prednisone (CHOP). Approximately 70% to 80% of patients will respond to their initial chemotherapy, duration of remissions last on the order of 2-3 years. Ultimately the majority of patients relapse. The discovery and clinical use of the anti-CD20 antibody, rituximab, has provided significant improvements in response and survival rate. The current standard of care for most patients is rituximab+CHOP (R-CHOP) or rituximab+CVP (R-CVP). Rituximab therapy has been shown to be efficacious in several types of NHL, and is currently approved as a first line treatment for both indolent (follicular lymphoma) and aggressive NHL (diffuse large B cell lymphoma). However, there are significant limitations of anti-CD20 monoclonal antibody (mAb), including primary resistance (50% response in relapsed indolent patients), acquired resistance (50% response rate upon re-treatment), rare complete response (2% complete response rate in relapsed population), and a continued pattern of relapse. Finally, many B cells do not express CD20, and thus many B-cell disorders are not treatable using anti-CD20 antibody therapy.

In addition to NHL there are several types of leukemias that result from dysregulation of B cells. Chronic lymphocytic leukemia (also known as “chronic lymphoid leukemia” or “CLL”), is a type of adult leukemia caused by an abnormal accumulation of B lymphocytes. In CLL, the malignant lymphocytes may look normal and mature, but they are not able to cope effectively with infection. CLL is the most common form of leukemia in adults. Men are twice as likely to develop CLL as women. However, the key risk factor is age. Over 75% of new cases are diagnosed in patients over age 50. More than 10,000 cases are diagnosed every year and the mortality is almost 5,000 a year (American Cancer Society, 2006; and SEER Cancer Statistics Review). CLL is an incurable disease but progresses slowly in most cases. Many people with CLL lead normal and active lives for many years. Because of its slow onset, early-stage CLL is generally not treated since it is believed that early CLL intervention does not improve survival time or quality of life. Instead, the condition is monitored over time. Initial CLL treatments vary depending on the exact diagnosis and the progression of the disease. There are dozens of agents used for CLL therapy. Combination chemotherapy regimens such as FCR (fludarabine, cyclophosphamide and rituximab), and BR (Ibrutinib and rituximab) are effective in both newly-diagnosed and relapsed CLL. Allogeneic bone marrow (stem cell) transplantation is rarely used as a first-line treatment for CLL due to its risk.

Another type of leukemia is Small lymphocytic lymphoma (“SLL”) that is considered a CLL variant that lacks the clonal lymphocytosis required for the CLL diagnosis, but otherwise shares pathological and immunophenotypic features (Campo et al., 2011). The definition of SLL requires the presence of lymphadenopathy and/or splenomegaly. Moreover, the number of B lymphocytes in the peripheral blood should not exceed 5×109/L. In SLL, the diagnosis should be confirmed by histopathologic evaluation of a lymph node biopsy whenever possible (Hallek et al., 2008). The incidence of SLL is approximately 25% of CLL in the US (Dores et al., 2007).

Another type of leukemia is acute lymphoblastic leukemia (ALL), also known as acute lymphocytic leukemia. ALL is characterized by the overproduction and continuous multiplication of malignant and immature white blood cells (also known as lymphoblasts) in the bone marrow. ‘Acute’ refers to the undifferentiated, immature state of the circulating lymphocytes (“blasts”), and that the disease progresses rapidly with life expectancy of weeks to months if left untreated. ALL is most common in childhood with a peak incidence of 4-5 years of age. Children of age 12-16 die more easily from it than others. Currently, at least 80% of childhood ALL are considered curable. Under 4,000 cases are diagnosed every year and the mortality is almost 1,500 a year (American Cancer Society, 2006; and SEER Cancer Statistics Review).

“Subject” or “patient” as used in this context refers to any mammal, including rodents, such as mouse or rat, and primates, such as cynomolgus monkey (Macaca fascicularis), rhesus monkey (Macaca mulatta) or humans (Homo sapiens). Preferably, the subject or patient is a primate, most preferably a human patient, even more preferably an adult human patient.

The terms “engineered” or “modified” as used herein includes manipulation of nucleic acids or polypeptides by synthetic means (e.g. by recombinant techniques, in vitro peptide synthesis, by enzymatic or chemical coupling of peptides or some combination of these techniques). Preferably, the antibodies or antibody fragments according to the present disclosure are engineered or modified to improve one or more properties, such as antigen binding, stability, half-life, effector function, immunogenicity, safety and the like. Preferably the antibodies or antibody fragments according to the present disclosure are engineered or modified to improve effector function, such as ADCC.

The “Fc region” is used to define the C-terminal region of an immunoglobulin heavy chain. The Fc region of an immunoglobulin generally comprises two constant domains, a CH2 domain and a CH3 domain. Unless otherwise specified herein, numbering of amino acid residues in the Fc region is according to the EU numbering system, also called the EU index, as described in Kabat et al., Sequences of Proteins of Immunological Interest, 5^{th} Ed. Public Health Service, National Institutes of Health, Bethesda, Md., 1991.

The antibody which is administered according to the present disclosure is administered to the patient in a therapeutically effective amount. A “therapeutically effective amount” refers to an amount sufficient to provide some improvement of the clinical manifestations of a given disease or disorder. The amount that is effective for a particular therapeutic purpose will depend on the severity of the disease or injury as well as on the weight and general state of the subject. It will be understood that determination of an appropriate dosage may be achieved, using routine experimentation, by constructing a matrix of values and testing different points in the matrix, all of which is within the ordinary skills of a trained physician or clinical scientist.

The terms “combination” or “pharmaceutical combination” refer to the administration of one therapy in addition to another therapy. As such, “in combination with” includes simultaneous (e.g., concurrent) and consecutive administration in any order. Each component can be administered at the same time or sequentially in any order at different points in time. Thus, each component can be administered separately but sufficiently closely in time so as to provide the desired therapeutic effect. By way of non-limiting example, a first therapy (e.g., agent, such as an anti-CD19 antibody) may be administered before (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 8 weeks, 8 weeks, 9 weeks, 10 weeks, 11 weeks, or 12 weeks), concurrently, or after (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 7 weeks, 8 weeks, 9 weeks, 10 weeks, 11 weeks, or 12 weeks or longer) the administration of a second therapy (e.g., pharmaceutical agent, such as anti-CD47 antibody) to a patient.

In some embodiments, the combined administration of an anti-CD19 antibody or antibody fragment thereof and a polypeptide that blocks the SIRPα-CD47 innate immune checkpoint (e.g. an anti-CD47 antibody) has a synergistic effect. The terms “synergy”, “synergism”, “synergistic” and “synergistic effect” which are used herein interchangeably, refer to an effect of compounds administered in combination where the effect is greater than the sum of the individual effects of each of the compounds administered alone.

A synergistic effect of a pharmaceutical combination disclosed herein can be determined by different methods. Examples of such methods include the methods of Chou et al., Clarke et al. and/or Webb et al. See Ting-Chao Chou, Theoretical Basis, Experimental Design, and Computerized Simulation of Synergism and Antagonism in Drug Combination Studies, Pharmacol Rev 58:621-681 (2006), which is incorporated by reference in its entirety. See also Clarke et al., Issues in experimental design and endpoint analysis in the study of experimental cytotoxic agents in vivo in breast cancer and other models, Breast Cancer Research and Treatment 46:255-278 (1997), which is incorporated by reference in its entirety. See also Webb, J. L. (1963) Enzyme and Metabolic Inhibitors, Academic Press, New York, which is incorporated by reference in its entirety.

### DETAILED DESCRIPTION OF THE INVENTION

### Anti-CD19 Antibodies

The use of a CD19 antibody in non-specific B cell lymphomas is discussed in WO2007076950 (US2007154473), which are both incorporated by reference. The use of a CD19 antibody in CLL, NHL and ALL is described in Scheuermann et al., CD19 Antigen in Leukemia and Lymphoma Diagnosis and Immunotherapy, Leukemia and Lymphoma, Vol. 18, 385-397 (1995), which is incorporated by reference in its entirety.

Additional antibodies specific for CD19 are described in WO2005012493 (U.S. Pat. No. 7,109,304), WO2010053716 (U.S. Ser. No. 12/266,999) (Immunomedics); WO2007002223 (US U.S. Pat. No. 8,097,703) (Medarex); WO2008022152 (Ser. No. 12/377,251) and WO2008150494 (Xencor), WO2008031056 (U.S. Ser. No. 11/852,106) (Medimmune); WO 2007076950 (U.S. Ser. No. 11/648,505) (Merck Patent GmbH); WO 2009/052431 (U.S. Ser. No. 12/253,895) (Seattle Genetics); and WO2010095031 (12/710,442) (Glenmark Pharmaceuticals), WO2012010562 and WO2012010561 (International Drug Development), WO2011147834 (Roche Glycart), and WO2012156455 (Sanofi), which are all incorporated by reference in their entireties.

A pharmaceutical composition includes an active agent, e.g. an antibody for therapeutic use in humans. A pharmaceutical composition may additionally include pharmaceutically acceptable carriers or excipients.

The dose of an antibody or antibody fragment comprised in a pharmaceutical composition according to the present disclosure administered to a patient may vary depending upon the age and the size of the patient, symptoms, conditions, route of administration, and the like. The dose is typically calculated according to body weight, or body surface area, age, or per individual. Depending on the severity of the condition, the frequency and the duration of the treatment can be adjusted. Effective dosages and schedules for administering pharmaceutical compositions comprising antibodies or antibody fragments specific for CD19 may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

The pharmaceutical composition may include dosage forms for intravenous, subcutaneous, intracutaneous and intramuscular injections, etc. These injectable preparations may be prepared by known methods. For example, the injectable preparations may be prepared, e.g., by dissolving, suspending or emulsifying the antibody or its salt described above in a sterile aqueous medium or an oily medium conventionally used for injections. Exemplary pharmaceutical compositions comprising an antibody or antibody fragment specific for CD19 that can be used in the context of the present disclosure are disclosed, e.g., in WO2008/022152 or WO2018/002031.

In certain ways of administration, e.g. intravenous administration, it is preferred to administer drug depending on the body weight of the patient. In other ways of administration, e.g. subcutaneous administration, it is preferred to administer drug at a flat, fixed does. The skilled person is aware of which dose in one way of administration is equivalent to another dose in another way of administration. The pharmacodynamics of a specific drug are typically taken into account in a reasoned decision to administer a drug in the required from and at a required, efficacious dose.

The antibody which is administered according to the present disclosure is administered to the patient in a therapeutically effective amount. A “therapeutically effective amount” refers to an amount sufficient to cure, alleviate or partially arrest the clinical manifestations of a given disease or disorder, i.e. NHL, and its complications. In certain embodiments the CD19 antibodies of the present disclosure are administered at 9 mg/kg. In alternative embodiments the CD19 antibodies of the present disclosure are administered at 12 mg/kg. In yet other embodiments CD19 antibodies of the present disclosure are administered at 15 mg/kg or more.

The antibody of the present disclosure can be administered at different time points and the treatment cycle may have a different length. The antibodies may be administered daily, every other day, three times a week, weekly or biweekly. The antibodies may also be administered over at least four weeks, over at least five weeks, over at least six weeks, over at least seven weeks, over at least eight weeks, over at least nine weeks, over at least ten weeks, over at least eleven weeks or over at least twelve weeks. In certain embodiments of the present disclosure the antibody is administered at least once weekly over at least eight weeks.

Polypeptides that Block the SIRPα-CD47 Innate Immune Checkpoint

CD47 is a broadly expressed transmembrane glycoprotein with a single Ig-like domain and five membrane spanning regions, which functions as a cellular ligand for SIRPoc with binding mediated through the NH2-terminal V-like domain of SIRPoc. SIRPoc is expressed primarily on myeloid cells, including macrophages, granulocytes, myeloid dendritic cells (DCs), mast cells, and their precursors, including hematopoietic stem cells. Structural determinants on SIRPoc that mediate CD47 binding are discussed by Lee et al. (2007) J. lmmunol. 179:7741-7750; Hatherley et al. (2008) Mol Cell. 31(2):266-77; Hatherley et al. (2007) J. B. C. 282:14567-75; and the role of SIRPoc cis dimerization in CD47 binding is discussed by Lee et al. (2010) J. B. C. 285:37953-63. In keeping with the role of CD47 to inhibit phagocytosis of normal cells, there is evidence that it is transiently upregulated on hematopoietic stem cells (HSCs) and progenitors just prior to and during their migratory phase, and that the level of CD47 on these cells determines the probability that they are engulfed in vivo.

Polypeptides that block the SIRPα-CD47 innate immune checkpoint refer to any polypeptide that reduces the binding of CD47 to SIRPα. Non-limiting examples of suitable SIRPα-CD47 innate immune checkpoint inhibitors include anti-SIRPα antibodies or antibody fragments, anti-CD47 antibodies or antibody fragments or polypeptidic SIRPα reagents. In some embodiments, a suitable SIRPα-CD47 innate immune checkpoint inhibitor (e.g. an anti-CD47 antibody, anti-SIRPα antibody, etc.) specifically binds CD47 or SIRPα to reduce the binding of CD47 to. SIRPα. In some embodiments, a suitable SIRPα-CD47 innate immune checkpoint inhibitor (e.g., an anti-SIRPα antibody, a soluble CD47 polypeptide, etc.) specifically binds SIRPα to reduce the binding of CD47 to SIRPα. A suitable SIRPα-CD47 innate immune checkpoint inhibitor that binds SIRPα does not activate SIRPα (e.g., in the SIRPα-expressing phagocytic cell). The efficacy of a suitable SIRPα-CD47 innate immune checkpoint inhibitor can be assessed by assaying the agent in an exemplary assay, where target cells are incubated in the presence or absence of the candidate agent. A SIRPα-CD47 innate immune checkpoint inhibitor (e.g. an anti-CD47 antibody, anti-SIRPα antibody, polypeptidic SIRPα reagent etc.) for use in the methods of the invention will up-regulate phagocytosis by at least 10% (e.g., at least 20%, at least 30%, at least 40%, at least 50%, at least 60%, at least 70%, at least 80%, at least 90%, at least 100%, at least 120%, at least 140%, at least 160%, at least 180%, or at least 200%) compared to phagocytosis in the absence of the agent. Similarly, an in vitro assay for levels of tyrosine phosphorylation of SIRPα will show a decrease in phosphorylation by at least 5% (e.g., at least 10%, at least 15%, at least 20%, at least 30%, at least 40%, at least 50%, at least 60%, at least 70%, at least 80%, at least 90%, or 100%) compared to phosphorylation observed in absence of the candidate agent.

In some embodiments, the anti-CD47 agent does not activate CD47 upon binding.

Some pathogens (e.g., pox viruses, Myxoma virus, Deerpox virus, swinepox virus, goatpox virus, sheeppox virus, etc.) express a CD47-analog (i.e., a CD47 mimic) (e.g., the M128L protein) that acts as a virulence factor to enable infection (Cameron et al, Virology. 2005 Jun. 20; 337(I):55-67), and some pathogens induce the expression of endogenous CD47 in the host cell. Cells infected with a pathogen that expresses a

CD47-analog may therefore express the pathogen-provided CD47 analog either exclusively or in combination with endogenous CD47. This mechanism allows the pathogen to increase CD47 expression (via expression of the CD47 analog) in the infected cell with or without increasing the level of endogenous CD47. In some embodiments, a polypeptidic SIRPα-CD47 innate immune checkpoint inhibitor (e.g., anti-CD47 antibody, a SIRPα reagent, a SIRPα antibody, a soluble CD47 polypeptide, etc.) can reduce the binding of a CD47 analog (i.e., a CD47 mimic) to SIRPα. In some cases, a polypeptidic SIRPα-CD47 innate immune checkpoint inhibitor (e.g., a SIRPα reagent, an anti-CD47 antibody, etc.) can bind a CD47 analog (i.e., a CD47 mimic) to reduce the binding of the CD47 analog to SIRPα. In some cases, a suitable SIRPα-CD47 innate immune checkpoint inhibitor (e.g., an anti-SIRPα antibody, a soluble CD47 polypeptide, etc.) can bind to SIRPα. A suitable SIRPα-CD47 innate immune checkpoint inhibitor that binds SIRPα does not activate SIRPα (e.g., in the SIRPα-expressing phagocytic cell). An anti-CD47 agent can be used in any of the methods provided herein when the pathogen is a pathogen that provides a CD47 analog. In other words the term “CD47,” as used herein, encompasses CD47 as well as polypeptidic CD47 analogs (i.e.,CD47 mimics).

In some embodiments, a subject SIRPα-CD47 innate immune checkpoint inhibitor is an antibody that specifically binds SIRPα (i.e., an anti-SIRPα antibody) and reduces the interaction between CD47 on one cell and SIRPα on another cell. Suitable anti-SIRPα antibodies can bind SIRPα without activating or stimulating signaling through SIRPα because activation of SIRPα would inhibit phagocytosis. Instead, suitable anti-SIRPα antibodies facilitate the preferential phagocytosis of inflicted cells over normal cells. Those cells that express higher levels of CD47 relative to other cells will be preferentially phagocytosed. Thus, a suitable anti-SIRPα antibody specifically binds SIRPα (without activating/stimulating enough of a signaling response to inhibit phagocytosis) and blocks an interaction between SIRPα and CD47. Suitable anti-SIRPα antibodies include fully human, humanized or chimeric versions of such antibodies. Humanized antibodies are especially useful for in vivo applications in humans due to their low antigenicity. Similarly caninized, felinized, etc. antibodies are especially useful for applications in dogs, cats, and other species respectively. Antibodies of interest include humanized antibodies, or caninized, felinized, equinized, bovinized, porcinized, etc., antibodies, and variants thereof.

In some embodiments, a subject polypeptide that blocks the SIRPα-CD47 innate immune checkpoint is a polypeptidic SIRPα reagent. In some embodiments the polypeptidic SIRPα reagent reduces the interaction between CD47 on one cell and SIRPα on another cell. A “polypeptidic SIRPα reagent” as used herein comprises the portion of SIRPα that is sufficient to bind CD47 at a recognizable affinity, which normally lies between the signal sequence and the transmembrane domain, or a fragment thereof that retains the binding activity. A suitable SIRPα reagent reduces (e.g., blocks, prevents, etc.) the interaction between the native proteins SIRPα and CD47. The SIRPα reagent will usually comprise at least a domain of SIRPα. In some embodiments, a SIRPα reagent is a fusion protein, e.g., fused in frame with a second polypeptide. In some embodiments, the second polypeptide is capable of increasing the size of the fusion protein, e.g., so that the fusion protein will not be cleared from the circulation rapidly. In some embodiments, the second polypeptide is part or whole of an immunoglobulin Fc region. The Fc region aids in phagocytosis by providing an “eat me” signal, which enhances the block of the “don't eat me” signal provided by the high affinity SIRPα reagent. In other embodiments, the second polypeptide is any suitable polypeptide that is substantially similar to Fc, e.g., providing increased size, multimerization domains, and/or additional binding or interaction with Ig molecules. In some embodiments the polypeptide that blocks the SIRPα-CD47 innate immune checkpoint is a SIRPαFc-fusion protein.

As used herein, an “anti-CD47 antibody” refers to any antibody or antibody fragment that reduces the binding of CD47 to a CD47 ligand such as SIRPα. In some embodiments, a suitable anti-CD47 antibody does not activate CD47 upon binding. Non-limiting examples of suitable antibodies include e.g. clones B6H12, 5F9, 8B6, and C3 (for example as described in International Patent Publication WO 2011/143624, herein specifically incorporated by reference). Suitable anti-CD47 antibodies include fully human humanized, or chimeric versions of antibodies. Humanized antibodies are especially useful for in vivo applications in humans due to their low antigenicity. Similarly caninized, felinized, etc. antibodies are especially useful for applications in dogs, cats, and other species respectively. Antibodies of interest include humanized antibodies, or caninized, felinized, equinized, bovinized, porcinized, etc., antibodies, and variants thereof.

An anti-CD47 antibody can be formulated in a pharmaceutical composition with a pharmaceutically acceptable excipient. An anti-CD47 antibody can be administered intravenously.

In some aspects, the anti-CD47 antibody competes for binding to CD47 with B6H12, 5F9, 8B6 or C3. In some aspects, the anti-CD47 binds to the same CD47 epitope as B6H12, 5F9, 8B6, or C3. In some aspects, the anti-CD47 antibody competes for binding to CD47 with B6H12. In some aspects, the anti-CD47 binds to the same CD47 epitope as B6H12.

Table 2 contains the sequence of the B6H12 antibody heavy and light chains and indicates the CDRs of the B6H12 antibody.

In some aspects, the anti-CD47 antibody binds to the same CD47 epitope as B6H12 wherein said B6H12 antibody or antibody fragment thereof comprises a heavy chain variable region comprising an HCDR1 region comprising the sequence GYGMS (SEQ ID NO: 14), an HCDR2 region comprising the sequence TITSGGTYTYYPDSVKG (SEQ ID NO: 15), and an HCDR3 region comprising the sequence SLAGNAMDY (SEQ ID NO: 16) and a light chain variable region comprising an LCDR1 region comprising the sequence RASQTISD (SEQ ID NO: 17), an LCDR2 region comprising the sequence FASQSIS (SEQ ID NO: 18), and an LCDR3 region comprising the sequence QNGHGFPRT (SEQ ID NO: 19). In one aspect said B6H12 antibody or antibody fragment thereof comprises a heavy chain variable region of

(SEQ ID NO: 20)
EVQLVESGGDLVKPGGSLKLSCAASGFTFSGYGMSWVRQTPDKRLEWVAT
ITSGGTYTYYPDSVKGRFTISRDNAKNTLYLQIDSLKSEDTAIYFCARSL
AGNAMDYWGQGTSVTVSS

and a light chain variable region of

(SEQ ID NO: 21)
DIVMTQSPATLSVTPGDRVSLSCRASQTISDYLHWYQQKSHESPRLLIKF
ASQSISGIPSRFSGSGSGSDFTLSINSVEPEDVGVYYCQNGHGFPRTFGG
GTKLEIK.

In some aspects, the anti-CD47 antibody competes for binding to CD47 with B6H12 wherein said B6H12 antibody or antibody fragment thereof comprises a heavy chain variable region comprising an HCDR1 region comprising the sequence GYGMS (SEQ ID NO: 14), an HCDR2 region comprising the sequence TITSGGTYTYYPDSVKG (SEQ ID NO: 15), and an HCDR3 region comprising the sequence SLAGNAMDY (SEQ ID NO: 16) and a light chain variable region comprising an LCDR1 region comprising the sequence RASQTISD (SEQ ID NO: 17), an LCDR2 region comprising the sequence FASQSIS (SEQ ID NO: 18), and an LCDR3 region comprising the sequence QNGHGFPRT (SEQ ID NO: 19). In one aspect said B6H12 antibody or antibody fragment thereof comprises a heavy chain variable region of

(SEQ ID NO: 20)
EVQLVESGGDLVKPGGSLKLSCAASGFTFSGYGMSWVRQTPDKRLEWVAT
ITSGGTYTYYPDSVKGRFTISRDNAKNTLYLQIDSLKSEDTAIYFCARSL
AGNAMDYWGQGTSVTVSS

and a light chain variable region of

(SEQ ID NO: 21)
DIVMTQSPATLSVTPGDRVSLSCRASQTISDYLHWYQQKSHESPRLLIKF
ASQSISGIPSRFSGSGSGSDFTLSINSVEPEDVGVYYCQNGHGFPRTFGG
GTKLEIK.

In some aspects, the anti-CD47 antibody competes for binding to CD47 with 5F9. In some aspects, the anti-CD47 binds to the same CD47 epitope as 5F9. In some aspects, the anti-CD47 antibody comprises an IgG4 Fc. In some aspects, the anti-CD47 antibody comprises or consists of 5F9.

In some embodiments, the methods described herein include administration of the anti-CD47 antibody 5F9. In some embodiments, the methods described herein include administration of an anti-CD47 antibody with sequences (light chain, heavy chain and/or CDR) at least 97%, at least 98%, at least 99% or 100% identical to the sequences of 5F9. Table 3 contains the sequences of the 5F9 antibody and variants thereof.

In some aspects, the anti-CD47 antibody binds to the same CD47 epitope as an antibody or antibody fragment that comprises a heavy chain variable region comprising an HCDR1 region comprising the sequence NYNMH (SEQ ID NO: 22), an HCDR2 region comprising the sequence TIYPGNDDTSYNQKFKD (SEQ ID NO: 23), and an HCDR3 region comprising the sequence GGYRAMDY (SEQ ID NO: 24) and a light chain variable region comprising an LCDR1 region comprising the sequence RSSQSIVYSNGNTYLG (SEQ ID NO: 25), an LCDR2 region comprising the sequence KVSNRFS (SEQ ID NO: 26), and an LCDR3 region comprising the sequence FQGSHVPYT (SEQ ID NO: 27). In one aspect said antibody or fragment thereof comprises a heavy chain variable region selected from the group consisting of SEQ ID NO: 28, SEQ ID NO: 30 and SEQ ID NO: 32 and a light chain variable region selected from the group consisting of SEQ ID NO: 29, SEQ ID NO: 31 and SEQ ID NO: 33. In one aspect said anti-CD47 antibody or fragment thereof comprises a heavy chain variable region of SEQ ID NO: 30 and a light chain variable region of SEQ ID NO: 31. In another aspect said anti-CD47 antibody or fragment thereof comprises a full heavy chain of SEQ ID NO: 34 and a full light chain of SEQ ID NO: 35.

In some aspects, the anti-CD47 antibody competes for binding to CD47 with an antibody or antibody fragment thereof that comprises a heavy chain variable region comprising an HCDR1 region comprising the sequence NYNMH (SEQ ID NO: 22), an HCDR2 region comprising the sequence TIYPGNDDTSYNQKFKD (SEQ ID NO: 23), and an HCDR3 region comprising the sequence GGYRAMDY (SEQ ID NO: 24) and a light chain variable region comprising an LCDR1 region comprising the sequence RSSQSIVYSNGNTYLG (SEQ ID NO: 25), an LCDR2 region comprising the sequence KVSNRFS (SEQ ID NO: 26), and an LCDR3 region comprising the sequence FQGSHVPYT (SEQ ID NO: 27). In one aspect said antibody or fragment thereof comprises a heavy chain variable region selected from the group consisting of SEQ ID NO: 28, SEQ ID NO: 30 and SEQ ID NO: 32 and a light chain variable region selected from the group consisting of SEQ ID NO: 29, SEQ ID NO: 31 and SEQ ID NO: 33.

In some aspects, the anti-CD47 antibody of fragment thereof comprises a heavy chain variable region comprising an HCDR1 region comprising the sequence NYNMH (SEQ ID NO: 22), an HCDR2 region comprising the sequence TIYPGNDDTSYNQKFKD (SEQ ID NO: 23), and an HCDR3 region comprising the sequence GGYRAMDY (SEQ ID NO: 24) and a light chain variable region comprising an LCDR1 region comprising the sequence RSSQSIVYSNGNTYLG (SEQ ID NO: 25), an LCDR2 region comprising the sequence KVSNRFS (SEQ ID NO: 26), and an LCDR3 region comprising the sequence FQGSHVPYT (SEQ ID NO: 27). In one aspect said anti-CD47 antibody or fragment thereof comprises a heavy chain variable region selected from the group consisting of SEQ ID NO: 28, SEQ ID NO: 30 and SEQ ID NO: 32 and a light chain variable region selected from the group consisting of SEQ ID NO: 29, SEQ ID NO: 31 and SEQ ID NO: 33. In one aspect the anti-CD47 antibody or fragment thereof comprises a heavy chain variable region of SEQ ID NO: 30 and a light chain variable region of SEQ ID NO: 31. In another aspect said anti-CD47 antibody or fragment thereof comprises a full heavy chain of SEQ ID NO: 34 and a full light chain of SEQ ID NO: 35.

In some aspects, the anti-CD47 antibody of fragment thereof comprises a heavy chain variable region comprising an HCDR1 region of the sequence NYNMH (SEQ ID NO: 22), an HCDR2 region of the sequence TIYPGNDDTSYNQKFKD (SEQ ID NO: 23), and an HCDR3 region of the sequence GGYRAMDY (SEQ ID NO: 24) and a light chain variable region comprising an LCDR1 region of the sequence RSSQSIVYSNGNTYLG (SEQ ID NO: 25), an LCDR2 region of the sequence KVSNRFS (SEQ ID NO: 26), and an LCDR3 region of the sequence FQGSHVPYT (SEQ ID NO: 27).

In one aspect said anti-CD47 antibody or fragment thereof comprises a heavy chain variable region of

(SEQ ID NO: 28)
QVQLVQSGAEVKKPGASVKVSCKASGYTFTNYNMHWVRQAPGQGLEWIGT
IYPGNDDTSYNQKFKDKATLTADKSTSTAYMELSSLRSEDTAVYYCARGG
YRAMDYWGQGTLVTVSS

and a light chain variable region of

(SEQ ID NO: 29)
DVVMTQSPLSLPVTPGEPASISCRSSQSIVYSNGNTYLGWYLQKPGQSPK
LLIYKVSNRFSGVPDRFSGSGSGTDFTLKISRVEAEDVGVYHCFQGSHVP
YTFGGGTKVEIK.

In one aspect said anti-CD47 antibody or fragment thereof comprises a heavy chain variable region of

(SEQ ID NO: 30)
QVQLVQSGAEVKKPGASVKVSCKASGYTFTNYNMHWVRQAPGQRLEWMGT
IYPGNDDTSYNQKFKDRVTITADTSASTAYMELSSLRSEDTAVYYCARGG
YRAMDYWGQGTLVTVSS

and a light chain variable region of

(SEQ ID NO: 31)
DIVMTQSPLSLPVTPGEPASISCRSSQSIVYSNGNTYLGWYLQKPGQSPQ
LLIYKVSNRFSGVPDRFSGSGSGTDFTLKISRVEAEDVGVYYCFQGSHVP
YTFGQGTKLEIK.

In one aspect said anti-CD47 antibody or fragment thereof comprises a heavy chain variable region of

(SEQ ID NO: 32)
QVQLVQSGAEVKKPGASVKVSCKASGYTFTNYNMHWVRQAPGQRLEWIGT
IYPGNDDTSYNQKFKDRATLTADKSASTAYMELSSLRSEDTAVYYCARGG
YRAMDYWGQGTLVTVSS

and a light chain variable region of

(SEQ ID NO: 33)
DVVMTQSPLSLPVTPGEPASISCRSSQSIVYSNGNTYLGWYLQKPGQSPQ
LLIYKVSNRFSGVPDRFSGSGSGTDFTLKISRVEAEDVGVYHCFQGSHVP
YTFGQGTKLEIK.

In one aspect said anti-CD47 antibody or fragment thereof comprises a heavy chain of

(SEQ ID NO: 34)
QVQLVQSGAEVKKPGASVKVSCKASGYTFTNYNMHWVRQAPGQRLEWMGT
IYPGNDDTSYNQKFKDRVTITADTSASTAYMELSSLRSEDTAVYYCARGG
YRAMDYWGQGTLVTVSSASTKGPSVFPLAPCSRSTSESTAALGCLVKDYF
PEPVTVSWNSGALTSGVHTFPAVLQSSGLYSLSSVVTVPSSSLGTKTYTC
NVDHKPSNTKVDKRVESKYGPPCPPCPAPEFLGGPSVFLFPPKPKDTLMI
SRTPEVTCVVVDVSQEDPEVQFNWYVDGVEVHNAKTKPREEQFNSTYRVV
SVLTVLHQDWLNGKEYKCKVSNKGLPSSIEKTISKAKGQPREPQVYTLPP
SQEEMTKNQVSLTCLVKGFYPSDIAVEWESNGQPENNYKTTPPVLDSDGS
FFLYSRLTVDKSRWQEGNVFSCSVMHEALHNHYTQKSLSLSLGK

and a light chain of

(SEQ ID NO: 35)
DIVMTQSPLSLPVTPGEPASISCRSSQSIVYSNGNTYLGWYLQKPGQSPQ
LLIYKVSNRFSGVPDRFSGSGSGTDFTLKISRVEAEDVGVYYCFQGSHVP
YTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC.

In some embodiments, a suitable anti-CD47 antibody does not activate CD47 upon binding. Non-limiting examples of suitable antibodies include clones B6H12, 5F9, 8B6, and C3 (for example as described in International Patent Publication WO 2011/143624, herein specifically incorporated by reference).

In some embodiments an anti-CD47 antibody comprises a human IgG Fc region, e.g. an IgGI, IgG2a, IgG2b, IgG3, IgG4 constant region. In one embodiment the IgG Fc region is an IgG4 constant region. The IgG4 hinge may be stabilized by the amino acid substitution S241P (see Angal et al. (1993) Mol. Immunol. 30(1): 105-108, herein specifically incorporated by reference).

### Methods of Treatment

Disclosed herein is a method of treating a human subject having cancer (e.g., a cancer identified as being a CD19+) or reducing the size of the cancer in the human subject, comprising:

(a) administering a polypeptide that blocks the SIRPα-CD47 innate immune checkpoint to the subject; and

(b) administering an anti-CD19 antibody to the subject.

Disclosed herein is a method of treating a human subject having a cancer (e.g., a cancer identified as being a CD19+) or reducing the size of the cancer in the human subject, comprising:

(a) administering a polypeptide that blocks the SIRPα-CD47 innate immune checkpoint to the subject at a dose of greater than or equal to 2 mg of antibody per kg of body weight; and

(b) administering an anti-CD19 antibody to the subject.

In an embodiment the present disclosure provides a method of treating a human subject having a cancer (e.g., a cancer identified as being a CD19+) or reducing the size of the cancer in the human subject, comprising:

(a) administering a polypeptide that blocks the SIRPα-CD47 innate immune checkpoint to the subject; and

(b) administering an anti-CD19 antibody to the subject,

wherein the cancer is a hematological cancer. In some aspects the hematological cancer is chronic lymphocytic leukemia (CLL), non-Hodgkin's lymphoma (NHL), small lymphocytic lymphoma (SLL) or acute lymphoblastic leukemia (ALL). In some other aspects the NHL is selected from the group consisting of follicular lymphoma, small lymphocytic lymphoma, mucosa-associated lymphoid tissue, marginal zone lymphoma, diffuse large B cell lymphoma, Burkitt's lymphoma, and mantle cell lymphoma.

In some aspects, the cancer is a CD19+ cancer. In some aspects, the CD19+ cancer is a hematological cancer. In some aspects, the hematological cancer is Non-Hodgkin's lymphoma (NHL). In some aspects, NHL is indolent lymphoma. In some aspects, indolent lymphoma is follicular lymphoma (FL). In some aspects, indolent lymphoma is marginal zone lymphoma. In some aspects, NHL is diffuse large B cell lymphoma (DLBCL). In some aspects, the CD19+ cancer is DLBCL, follicular lymphoma, marginal zone lymphoma, mantle cell lymphoma, chronic lymphocytic leukemia/small lymphocytic leukemia, Waldenstrom's macroglobulinemia/lymphoplasmacytic lymphoma, primary mediastinal B-cell lymphoma, Burkitt's lymphoma, B-cell lymphoma unclassified, B-cell acute lymphoblastic leukemia, or post-transplant lymphoproliferative disease (PTLD), optionally wherein the CD19+ cancer is classified based on histopathology, flow cytometry, molecular classification, one or more equivalent assays, or a combination thereof. In some aspects, the CD19+ cancer is double hit lymphoma. In some aspects, the CD19+ cancer is myc-rearranged lymphoma.

In some aspects, the subject is relapsed or refractory to at least 1, 2, 3, 4, 5, 6, 7, 8, 9, 10 or greater than 10 prior lines of cancer therapy. In some aspects, the subject is refractory to rituximab. In some aspects, rituximab refractory status is a failure to respond to, or progression during, any previous rituximab-containing regimen, or progression within 6 months of the last rituximab dose.

In an embodiment the present disclosure provides a method of treating a human subject having a cancer (e.g., a cancer identified as being a CD19+) or reducing the size of the cancer in the human subject, comprising:

(a) administering a polypeptide that blocks the SIRPα-CD47 innate immune checkpoint to the subject; and

(b) administering an anti-CD19 antibody to the subject,

wherein said anti-CD19 antibody or antibody fragment thereof and the polypeptide that blocks the SIRPα-CD47 innate immune checkpoint are administered in a separate manner. In another aspect said anti-CD19 antibody or antibody fragment thereof and the polypeptide that blocks the SIRPα-CD47 innate immune checkpoint are administered in a simultaneous manner.

In another embodiment the present disclosure provides a method of treating a human subject having a cancer (e.g., a cancer identified as being a CD19+) or reducing the size of the cancer in the human subject, comprising:

(a) administering a polypeptide that blocks the SIRPα-CD47 innate immune checkpoint to the subject; and

(b) administering an anti-CD19 antibody to the subject,

wherein said anti-CD19 antibody or antibody fragment thereof wherein said anti-CD19 antibody or antibody fragment thereof comprises a heavy chain variable region comprising an HCDR1 region comprising the sequence SYVMH (SEQ ID NO: 1), an HCDR2 region comprising the sequence NPYNDG (SEQ ID NO: 2), and an HCDR3 region comprising the sequence GTYYYGTRVFDY (SEQ ID NO: 3) and a light chain variable region comprising an LCDR1 region comprising the sequence RSSKSLQNVNGNTYLY (SEQ ID NO: 4), an LCDR2 region comprising the sequence RMSNLNS (SEQ ID NO: 5), and an LCDR3 region comprising the sequence MQHLEYPIT (SEQ ID NO: 6). In one aspect said anti-CD19 antibody or antibody fragment thereof comprises a heavy chain variable region of

(SEQ ID NO: 7)
EVQLVESGGGLVKPGGSLKLSCAASGYTFTSYVMHVVVRQAPGKGLEWIG
YINPYNDGTKYNEKFQGRVTISSDKSISTAYMELSSLRSEDTAMYYCARG
TYYYGTRVFDYWGQGTLVTVSS

and a light chain variable region of

(SEQ ID NO: 8)
DIVMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIK.

In a further aspect said anti-CD19 antibody comprises a heavy chain of

(SEQ ID NO: 11)
EVQLVESGGGLVKPGGSLKLSCAASGYTFTSYVMHWVRQAPGKGLEWIGY
INPYNDGTKYNEKFQGRVTISSDKSISTAYMELSSLRSEDTAMYYCARGT
YYYGTRVFDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPDVFLFPPK
PKDTLMISRTPEVICVVVDVSHEDPEVQFNWYVDGVEVHNAKTKPREEQF
NSTFRVVSVLTVVHQDWLNGKEYKCKVSNKALPAPEEKTISKTKGQPREP
QVYTLPPSREEMTKNQVSLTCLVKGFYPSDIAVEWESNGQPENNYKTTPP
MLDSDGSFFLYSKLTVDKSRWQQGNVFSCSVMHEALHNHYTQKSLSLSPG
K.

In a further aspect said anti-CD19 antibody or antibody fragment thereof further comprises a light chain of

(SEQ ID NO: 12)
DIVMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC.

In another embodiment the present disclosure provides a method of treating a human subject having a cancer (e.g., a cancer identified as being a CD19+) or reducing the size of the cancer in the human subject, comprising:

(a) administering a polypeptide that blocks the SIRPα-CD47 innate immune checkpoint to the subject; and

(b) administering an anti-CD19 antibody to the subject, wherein said polypeptide that blocks the SIRPα-CD47 innate immune checkpoint is an antibody or antibody fragment that specifically binds to human CD47 or human SIRPα. In another aspect the polypeptide that blocks the SIRPα-CD47 innate immune checkpoint is a polypeptidic SIRPα reagent. In a further aspect the polypeptide that blocks the SIRPα-CD47 innate immune checkpoint is a SIRPαFc fusion protein.

In another embodiment the present disclosure provides a method of treating a human subject having a cancer (e.g., a cancer identified as being a CD19+) or reducing the size of the cancer in the human subject, comprising:

(a) administering an anti-CD47 antibody to the subject; and

(b) administering an anti-CD19 antibody to the subject.

In some aspects, the anti-CD47 antibody competes for binding to CD47 with B6H12, 5F9, 8B6 or C3. In some aspects, the anti-CD47 binds to the same CD47 epitope as B6H12, 5F9, 8B6, or C3. In some aspects, the anti-CD47 antibody competes for binding to CD47 with B6H12. In some aspects, the anti-CD47 binds to the same CD47 epitope as B6H12.

Table 2 contains the sequence of the B6H12 antibody heavy and light chains and indicates the CDRs of the B6H12 antibody.

In some aspects, the anti-CD47 antibody binds to the same CD47 epitope as B6H12 wherein said B6H12 antibody or antibody fragment thereof comprises a heavy chain variable region comprising an HCDR1 region comprising the sequence GYGMS (SEQ ID NO: 14), an HCDR2 region comprising the sequence TITSGGTYTYYPDSVKG (SEQ ID NO: 15), and an HCDR3 region comprising the sequence SLAGNAMDY (SEQ ID NO: 16) and a light chain variable region comprising an LCDR1 region comprising the sequence RASQTISD (SEQ ID NO: 17), an LCDR2 region comprising the sequence FASQSIS (SEQ ID NO: 18), and an LCDR3 region comprising the sequence QNGHGFPRT (SEQ ID NO: 19). In one aspect said B6H12 antibody or antibody fragment thereof comprises a heavy chain variable region of

(SEQ ID NO: 20)
EVQLVESGGDLVKPGGSLKLSCAASGFTFSGYGMSWVRQTPDKRLEWVAT
ITSGGTYTYYPDSVKGRFTISRDNAKNTLYLQIDSLKSEDTAIYFCARSL
AGNAMDYWGQGTSVTVSS

and a light chain variable region of

(SEQ ID NO: 21)
DIVMTQSPATLSVTPGDRVSLSCRASQTISDYLHWYQQKSHESPRLLIKF
ASQSISGIPSRFSGSGSGSDFTLSINSVEPEDVGVYYCQNGHGFPRTFGG
GTKLEIK.

In some aspects, the anti-CD47 antibody competes for binding to CD47 with B6H12 wherein said B6H12 antibody or antibody fragment thereof comprises a heavy chain variable region comprising an HCDR1 region comprising the sequence GYGMS (SEQ ID NO: 14), an HCDR2 region comprising the sequence TITSGGTYTYYPDSVKG (SEQ ID NO: 15), and an HCDR3 region comprising the sequence SLAGNAMDY (SEQ ID NO: 16) and a light chain variable region comprising an LCDR1 region comprising the sequence RASQTISD (SEQ ID NO: 17), an LCDR2 region comprising the sequence FASQSIS (SEQ ID NO: 18), and an LCDR3 region comprising the sequence QNGHGFPRT (SEQ ID NO: 19). In one aspect said B6H12 antibody or antibody fragment thereof comprises a heavy chain variable region of

(SEQ ID NO: 20)
EVQLVESGGDLVKPGGSLKLSCAASGFTFSGYGMSWVRQTPDKRLEWVAT
ITSGGTYTYYPDSVKGRFTISRDNAKNTLYLQIDSLKSEDTAIYFCARSL
AGNAMDYWGQGTSVTVSS

and a light chain variable region of

(SEQ ID NO: 21)
DIVMTQSPATLSVTPGDRVSLSCRASQTISDYLHWYQQKSHESPRLLIKF
ASQSISGIPSRFSGSGSGSDFTLSINSVEPEDVGVYYCQNGHGFPRTFGG
GTKLEIK.

In some aspects, the anti-CD47 antibody competes for binding to CD47 with 5F9. In some aspects, the anti-CD47 binds to the same CD47 epitope as 5F9. In some aspects, the anti-CD47 antibody comprises an IgG4 Fc. In some aspects, the anti-CD47 antibody comprises or consists of 5F9.

In some embodiments, the methods described herein include administration of the anti-CD47 antibody 5F9. In some embodiments, the methods described herein include administration of an anti-CD47 antibody with sequences (light chain, heavy chain and/or CDR) at least 97%, at least 98%, at least 99% or 100% identical to the sequences of 5F9. Table 3 contains the sequences of the 5F9 antibody and variants thereof.

In some aspects, the anti-CD47 antibody binds to the same CD47 epitope as an antibody or antibody fragment that comprises a heavy chain variable region comprising an HCDR1 region comprising the sequence NYNMH (SEQ ID NO: 22), an HCDR2 region comprising the sequence TIYPGNDDTSYNQKFKD (SEQ ID NO: 23), and an HCDR3 region comprising the sequence GGYRAMDY (SEQ ID NO: 24) and a light chain variable region comprising an LCDR1 region comprising the sequence RSSQSIVYSNGNTYLG (SEQ ID NO: 25), an LCDR2 region comprising the sequence KVSNRFS (SEQ ID NO: 26), and an LCDR3 region comprising the sequence FQGSHVPYT (SEQ ID NO: 27). In one aspect said antibody or fragment thereof comprises a heavy chain variable region selected from the group consisting of SEQ ID NO: 28, SEQ ID NO: 30 and SEQ ID NO: 32 and a light chain variable region selected from the group consisting of SEQ ID NO: 29, SEQ ID NO: 31 and SEQ ID NO: 33.

In some aspects, the anti-CD47 antibody competes for binding to CD47 with an antibody or antibody fragment thereof that comprises a heavy chain variable region comprising an HCDR1 region comprising the sequence NYNMH (SEQ ID NO: 22), an HCDR2 region comprising the sequence TIYPGNDDTSYNQKFKD (SEQ ID NO: 23), and an HCDR3 region comprising the sequence GGYRAMDY (SEQ ID NO: 24) and a light chain variable region comprising an LCDR1 region comprising the sequence RSSQSIVYSNGNTYLG (SEQ ID NO: 25), an LCDR2 region comprising the sequence KVSNRFS (SEQ ID NO: 26), and an LCDR3 region comprising the sequence FQGSHVPYT (SEQ ID NO: 27). In one aspect said antibody or fragment thereof comprises a heavy chain variable region selected from the group consisting of SEQ ID NO: 28, SEQ ID NO: 30 and SEQ ID NO: 32 and a light chain variable region selected from the group consisting of SEQ ID NO: 29, SEQ ID NO: 31 and SEQ ID NO: 33.

In some aspects, the anti-CD47 antibody of fragment thereof comprises a heavy chain variable region comprising an HCDR1 region comprising the sequence NYNMH (SEQ ID NO: 22), an HCDR2 region comprising the sequence

TIYPGNDDTSYNQKFKD (SEQ ID NO: 23), and an HCDR3 region comprising the sequence GGYRAMDY (SEQ ID NO: 24) and a light chain variable region comprising an LCDR1 region comprising the sequence RSSQSIVYSNGNTYLG (SEQ ID NO: 25), an LCDR2 region comprising the sequence KVSNRFS (SEQ ID NO: 26), and an LCDR3 region comprising the sequence FQGSHVPYT (SEQ ID NO: 27). In one aspect said anti-CD47 antibody or fragment thereof comprises a heavy chain variable region selected from the group consisting of SEQ ID NO: 28, SEQ ID NO: 30 and SEQ ID NO: 32 and a light chain variable region selected from the group consisting of SEQ ID NO: 29, SEQ ID NO: 31 and SEQ ID NO: 33.

In one aspect the anti-CD47 antibody or fragment thereof comprises a heavy chain variable region of SEQ ID NO: 30 and a light chain variable region of SEQ ID NO: 31. In another aspect said anti-CD47 antibody or fragment thereof comprises a full heavy chain of SEQ ID NO: 34 and a full light chain of SEQ ID NO: 35.

In some aspects, the anti-CD47 antibody of fragment thereof comprises a heavy chain variable region comprising an HCDR1 region of the sequence NYNMH (SEQ ID NO: 22), an HCDR2 region of the sequence TIYPGNDDTSYNQKFKD (SEQ ID NO: 23), and an HCDR3 region of the sequence GGYRAMDY (SEQ ID NO: 24) and a light chain variable region comprising an LCDR1 region of the sequence RSSQSIVYSNGNTYLG (SEQ ID NO: 25), an LCDR2 region of the sequence KVSNRFS (SEQ ID NO: 26), and an LCDR3 region of the sequence FQGSHVPYT (SEQ ID NO: 27).

In one aspect said anti-CD47 antibody or fragment thereof comprises a heavy chain variable region of

(SEQ ID NO: 28)
QVQLVQSGAEVKKPGASVKVSCKASGYTFTNYNMHWVRQAPGQGLEWIGT
IYPGNDDTSYNQKFKDKATLTADKSTSTAYMELSSLRSEDTAVYYCARGG
YRAMDYWGQGTLVTVSS

and a light chain variable region of

(SEQ ID NO: 29)
DVVMTQSPLSLPVTPGEPASISCRSSQSIVYSNGNTYLGWYLQKPGQSPK
LLIYKVSNRFSGVPDRFSGSGSGTDFTLKISRVEAEDVGVYHCFQGSHVP
YTFGGGTKVEIK.

In one aspect said anti-CD47 antibody or fragment thereof comprises a heavy chain variable region of

(SEQ ID NO: 30)
QVQLVQSGAEVKKPGASVKVSCKASGYTFTNYNMHWVRQAPGQRLEWMGT
IYPGNDDTSYNQKFKDRVTITADTSASTAYMELSSLRSEDTAVYYCARGG
YRAMDYWGQGTLVTVSS

and a light chain variable region of

(SEQ ID NO: 31)
DIVMTQSPLSLPVTPGEPASISCRSSQSIVYSNGNTYLGWYLQKPGQSPQ
LLIYKVSNRFSGVPDRFSGSGSGTDFTLKISRVEAEDVGVYYCFQGSHVP
YTFGQGTKLEIK.

In one aspect said anti-CD47 antibody or fragment thereof comprises a heavy chain variable region of

(SEQ ID NO: 32)
QVQLVQSGAEVKKPGASVKVSCKASGYTFTNYNMHVVVRQAPGQRLEWIG
TIYPGNDDTSYNQKFKDRATLTADKSASTAYMELSSLRSEDTAVYYCARG
GYRAMDYWGQGTLVTVSS

and a light chain variable region of

(SEQ ID NO: 33)
DVVMTQSPLSLPVTPGEPASISCRSSQSIVYSNGNTYLGWYLQKPGQSPQ
LLIYKVSNRFSGVPDRFSGSGSGTDFTLKISRVEAEDVGVYHCFQGSHVP
YTFGQGTKLEIK.

In one aspect said anti-CD47 antibody or fragment thereof comprises a heavy chain of

(SEQ ID NO: 34)
QVQLVQSGAEVKKPGASVKVSCKASGYTFTNYNMHWVRQAPGQRLEWMGT
IYPGNDDTSYNQKFKDRVTITADTSASTAYMELSSLRSEDTAVYYCARGG
YRAMDYWGQGTLVTVSSASTKGPSVFPLAPCSRSTSESTAALGCLVKDYF
PEPVTVSWNSGALTSGVHTFPAVLQSSGLYSLSSVVTVPSSSLGTKTYTC
NVDHKPSNTKVDKRVESKYGPPCPPCPAPEFLGGPSVFLFPPKPKDTLMI
SRTPEVTCVVVDVSQEDPEVQFNWYVDGVEVHNAKTKPREEQFNSTYRVV
SVLTVLHQDWLNGKEYKCKVSNKGLPSSIEKTISKAKGQPREPQVYTLPP
SQEEMTKNQVSLTCLVKGFYPSDIAVEWESNGQPENNYKTTPPVLDSDGS
FFLYSRLTVDKSRWQEGNVFSCSVMHEALHNHYTQKSLSLSLGK

and a light chain of

(SEQ ID NO: 35)
DIVMTQSPLSLPVTPGEPASISCRSSQSIVYSNGNTYLGWYLQKPGQSPQ
LLIYKVSNRFSGVPDRFSGSGSGTDFTLKISRVEAEDVGVYYCFQGSHVP
YTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC.

In some embodiments, a suitable anti-CD47 antibody does not activate CD47 upon binding. Non-limiting examples of suitable antibodies include clones B6H12, 5F9, 8B6, and C3 (for example as described in International Patent Publication WO 2011/143624, herein specifically incorporated by reference).

In some embodiments an anti-CD47 antibody comprises a human IgG Fc region, e.g. an IgGI, IgG2a, IgG2b, IgG3, IgG4 constant region. In one embodiment the IgG Fc region is an IgG4 constant region. The IgG4 hinge may be stabilized by the amino acid substitution S241P (see Angal et al. (1993) Mol. Immunol. 30(1): 105-108, herein specifically incorporated by reference).

Methods are provided for treating a subject with a therapeutically effective dose of an antibody or antibody fragment specific for CD19 and a polypeptide that blocks the SIRPα-CD47 innate immune checkpoint.

Suitable administration of a therapeutically effective dose can entail administration of a single dose, or can entail administration of doses daily, semi-weekly, weekly, once every two weeks, once a month, annually, etc. In some cases, a therapeutically effective dose is administered as two or more doses of escalating concentration (i.e., increasing doses), where (i) all of the doses are therapeutic doses, or where (ii) a sub-therapeutic dose (or two or more sub-therapeutic doses) is initially given and therapeutic doses are achieved by said escalation.

An initial dose of a an antibody or antibody fragment specific for CD19 or a polypeptide that blocks the SIRPα-CD47 innate immune checkpoint, may lead to hemagglutination for a period of time immediately following infusion. Without being bound by the theory, it is believed that the initial dose of a multivalent CD47 binding agent may cause cross-linking of RBC bound to the agent. In certain embodiments of the invention the polypeptide that blocks the SIRPα-CD47 innate immune checkpoint is infused to a patient in an initial dose, and optionally in subsequent doses, over a period of time and/or concentration that reduces the possibility of hematologic microenvironments where there is a high local concentration of RBC and the agent.

In some embodiments, an initial dose of a CD47 binding agent is infused over a period of at least about 2 hours, at least about 2.5 hours, at least about 3 hours, at least about 3.5 hours, at least about 4 hours, at least about 4.5 hours, at least about 5 hours, at least about 6 hours or more. In some embodiments an initial dose is infused over a period of time from about 2.5 hours to about 6 hours; for example from about 3 hours to about 4 hours. In some such embodiments, the dose of agent in the infusate is from about 0.05 mg/ml to about 0.5 mg/ml; for example from about 0.1 mg/ml to about 0.25 mg/ml.

Dosage and frequency may vary depending on the half-life of the anti-CD47 antibody and/or the additional agent (e.g. an anti-CD19 antibody) in the patient. It will be understood by one of skill in the art that such guidelines will be adjusted for the molecular weight of the active agent, e.g. in the use of antibody fragments, in the use of antibody conjugates, in the use of SIRPα reagents, in the use of soluble CD47 peptides etc. The dosage may also be varied for localized administration, e.g. intranasal, inhalation, etc., or for systemic administration, e.g. i.m, i.p., i.v., s.c., and the like.

In certain embodiments of the invention, the anti-CD47 antibody is infused to a patient in an initial dose, and optionally in subsequent doses, over a period of time and/or concentration that reduces the possibility of hematologic microenvironments where there is a high local concentration of RBC and the agent. In some embodiments of the invention, an initial dose of the anti-CD47 antibody is infused over a period of at least about 2 hours, at least about 2.5 hours, at least about 3 hours, at least about 3.5 hours, at least about 4 hours, at least about 4.5 hours, at least about 5 hours, at least about 6 hours or more. In some embodiments an initial dose is infused over a period of time from about 2.5 hours to about 6 hours; for example from about 3 hours to about 4 hours. In some such embodiments, the dose of agent in the infusate is from about 0.05 mg/ml to about 0.5 mg/ml; for example from about 0.1 mg/ml to about 0.25 mg/ml.

### Methods of Administration

One or multiple polypeptides that blocks the SIRPα-CD47 innate immune checkpoint and the antibody or antibody fragment specific for CD19 can be administered to a subject in any order or simultaneously. If simultaneously, the polypeptide that blocks the SIRPα-CD47 innate immune checkpoint and the antibody or antibody fragment specific for CD19 can be provided in a single, unified form, such as an intravenous or subcutaneous injection, or in multiple forms, for example, as multiple intravenous or subcutaneous infusions, s.c, injections. The polypeptide that blocks the SIRPα-CD47 innate immune checkpoint and the antibody or antibody fragment specific for CD19 can be packed together or separately, in a single package or in a plurality of packages. One or all of the polypeptides that blocks the SIRPα-CD47 innate immune checkpoint and the antibody or antibody fragment specific for CD19 can be given in multiple doses. If not simultaneous, the timing between the multiple doses may vary to as much as about a week, a month, two months, three months, four months, five months, six months, or about a year. The polypeptide that blocks the SIRPα-CD47 innate immune checkpoint and/or the antibody or antibody fragment specific for CD19 of the disclosure, and pharmaceutical compositions comprising the same, can be packaged as a kit. A kit may include instructions (e.g., written instructions) on the use of the polypeptide that blocks the SIRPα-CD47 innate immune checkpoint and the antibody or antibody fragment specific for CD19 and compositions comprising the same.

In some cases, a method of treating a cancer comprises administering to a subject a therapeutically-effective amount of the polypeptide that blocks the SIRPα-CD47 innate immune checkpoint and the antibody or antibody fragment specific for CD19, wherein the administration treats the cancer. In some embodiments the therapeutically-effective amount of the polypeptide that blocks the SIRPα-CD47 innate immune checkpoint and the antibody or antibody fragment specific for CD19 is administered for at least about 10 seconds, 30 seconds, 1 minute, 10 minutes, 30 minutes, 1 hour, 2 hours, 3 hours, 4 hours, 5 hours, 8 hours, 12 hours, 24 hours, 2 days, 3 days, 4 days, 5 days, 6 days, 1 week, 2 weeks, 3 weeks, 1 month, 2 months, 3 months, 4 months, 5 months, 6 months, or 1 year.

The methods described herein include administration of a therapeutically effective dose of compositions, i.e., a therapeutically effective dose of a polypeptide that blocks the SIRPα-CD47 innate immune checkpoint and the antibody or antibody fragment specific for CD19 Compositions are administered to a patient in an amount sufficient to substantially ablate targeted cells, as described above. Single or multiple administrations of the compositions may be administered depending on the dosage and frequency as needed and tolerated by the patient. The particular dose used for a treatment will depend upon the medical condition and history of the mammal, as well as other factors such as age, weight, gender, administration route, efficiency, etc.

### Embodiments

The present disclosure provides a pharmaceutical combination, comprising an antibody or antibody fragment specific for CD19 and a polypeptide that blocks the SIRPα-CD47 innate immune checkpoint for use in the treatment of cancer.

In one aspect the present disclosure provides a pharmaceutical combination, comprising an antibody or antibody fragment specific for CD19 and a polypeptide that blocks the SIRPα-CD47 innate immune checkpoint for use in the treatment of cancer wherein the cancer is a hematological cancer. In one embodiment the hematological cancer is chronic lymphocytic leukemia (CLL), non-Hodgkin's lymphoma (NHL), small lymphocytic lymphoma (SLL) or acute lymphoblastic leukemia (ALL). In another embodiment the hematological cancer is non-Hodgkin's lymphoma (NHL). In a further embodiment the non-Hodgkin's lymphoma is selected from the group consisting of follicular lymphoma, small lymphocytic lymphoma, mucosa-associated lymphoid tissue, marginal zone lymphoma, diffuse large B cell lymphoma, Burkitt's lymphoma and mantle cell lymphoma.

In certain embodiments the present disclosure provides a pharmaceutical combination, comprising an antibody or antibody fragment specific for CD19 and a polypeptide that blocks the SIRPα-CD47 innate immune checkpoint for use in the treatment of cancer wherein said antibody or antibody fragment specific for CD19 is administered at 9 mg/kg. In alternative embodiments the antibody or antibody fragment specific for CD19 is administered at 12 mg/kg. In yet other embodiments at 15 mg/kg or more.

In embodiments, the antibody or antibody fragment specific for CD19 has a cytotoxic activity. In embodiments, the antibody or antibody fragment specific for CD19 comprises a constant region having ADCC inducing activity. In embodiments, the antibody specific for CD19 induces ADCC.

In certain embodiments the present disclosure provides a pharmaceutical combination, comprising an antibody or antibody fragment specific for CD19 and a polypeptide that blocks the SIRPα-CD47 innate immune checkpoint for use in the treatment of cancer wherein the components of the combination, the antibody or antibody fragment specific for CD19 and the polypeptide that blocks the SIRPα-CD47 innate immune checkpoint are administered separately. In an embodiment, the polypeptide that blocks the SIRPα-CD47 innate immune checkpoint are administered prior to administration of the antibody or antibody fragment specific for CD19. In an embodiment, the antibody or antibody fragment specific for CD19 are administered prior to administration of the polypeptide that blocks the SIRPα-CD47 innate immune checkpoint. In embodiments, the components of the combination are administered at a time where both components (drugs) are active in the patient at the same time. In embodiments, the components of the combination are administered together, simultaneously, separately or subsequently, either physically or in time. In embodiments, the components of the combination are administered simultaneously.

In certain embodiments the present disclosure provides a pharmaceutical combination, comprising an antibody or antibody fragment specific for CD19 and a polypeptide that blocks the SIRPα-CD47 innate immune checkpoint for use in the treatment of cancer wherein the anti-CD19 antibody is administered weekly, bi-weekly or monthly.

In certain embodiments the present disclosure provides a pharmaceutical combination, comprising an antibody or antibody fragment specific for CD19 and a polypeptide that blocks the SIRPα-CD47 innate immune checkpoint for use in the treatment of cancer wherein said antibody or antibody fragment specific for CD19 is administered in a concentration of 12mg/kg.

In certain embodiments the present disclosure provides a pharmaceutical combination, comprising an antibody or antibody fragment specific for CD19 and a polypeptide that blocks the SIRPα-CD47 innate immune checkpoint for use in the treatment of cancer wherein said antibody or antibody fragment specific for CD19 is administered weekly, bi-weekly or monthly after a first administration on Day 1 and wherein the polypeptide that blocks the SIRPα-CD47 innate immune checkpoint is administered for the first time on Day 8. In a further embodiment the anti-CD19 antibody or antibody fragment thereof after a first administration on Day 1 is administered weekly for the first 3 months and bi-weekly for at least the next 3 months.

In one aspect the present disclosure provides an anti-CD19 antibody or antibody fragment thereof for use in the treatment of hematological cancer patients, wherein said hematologic cancer patient has non-Hodgkin's lymphoma and wherein said anti-CD19 antibody or antibody fragment thereof is administered in combination with a polypeptide that blocks the SIRPα-CD47 innate immune checkpoint.

In one aspect the present disclosure provides an anti-CD19 antibody or antibody fragment thereof for use in the treatment of hematological cancer patients, wherein said hematologic cancer patient has non-Hodgkin's lymphoma and wherein said anti-CD19 antibody or antibody fragment thereof is administered in combination with a polypeptide that blocks the SIRPα-CD47 innate immune checkpoint. In one embodiment the hematological cancer patient has non-Hodgkin's lymphoma, wherein the non-Hodgkin's lymphoma is selected from the group consisting of follicular lymphoma, small lymphocytic lymphoma, mucosa-associated lymphoid tissue, marginal zone lymphoma, diffuse large B cell lymphoma, Burkitt's lymphoma and mantle cell lymphoma.

In an embodiment the anti-CD19 antibody or antibody fragment thereof for use in the treatment of hematological cancer patients in combination with a polypeptide that blocks the SIRPα-CD47 innate immune checkpoint comprises an HCDR1 region comprising the sequence SYVMH (SEQ ID NO: 1), an HCDR2 region comprising the sequence NPYNDG (SEQ ID NO: 2), an HCDR3 region comprising the sequence GTYYYGTRVFDY (SEQ ID NO: 3), an LCDR1 region comprising the sequence RSSKSLQNVNGNTYLY (SEQ ID NO: 4), an LCDR2 region comprising the sequence RMSNLNS (SEQ ID NO: 5), and an LCDR3 region comprising the sequence MQHLEYPIT (SEQ ID NO: 6).

In a further embodiment the anti-CD19 antibody or antibody fragment thereof for use in the treatment of hematological cancer patients in combination with a polypeptide that blocks the SIRPα-CD47 innate immune checkpoint comprises a variable heavy chain of the sequence

(SEQ ID NO: 7)
EVQLVESGGGLVKPGGSLKLSCAASGYTFTSYVMHWVRQAPGKGLEWIGY
INPYNDGTKYNEKFQGRVTISSDKSISTAYMELSSLRSEDTAMYYCARGT
YYYGTRVFDYWGQGTLVTVSS

and a variable light chain of the sequence

(SEQ ID NO: 8)
DIVMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIK.

In another embodiment of the present disclosure the anti-CD19 antibody or antibody fragment thereof is a human, humanized or chimeric antibody or antibody fragment. In another embodiment of the present disclosure the anti-CD19 antibody or antibody fragment thereof is of the IgG isotype. In another embodiment the antibody or antibody fragment is IgG1, IgG2 or IgG1/IgG2 chimeric. In another embodiment of the present disclosure the isotype of the anti-CD19 antibody is engineered to enhance antibody-dependent cell-mediated cytotoxicity. In another embodiment the heavy chain constant region of the anti-CD19 antibody comprises amino acids 239D and 332E, wherein the Fc numbering is according to the EU index as in Kabat. In another embodiment the antibody is IgG1, IgG2 or IgG1/IgG2 and the chimeric heavy chain constant region of the anti-CD19 antibody comprises amino acids 239D and 332E, wherein the Fc numbering is according to the EU index as in Kabat.

In a further embodiment the anti-CD19 antibody for use in the treatment of hematological cancer patients in combination with a polypeptide that blocks the SIRPα-CD47 innate immune checkpoint comprises a heavy chain having the sequence

(SEQ ID NO: 11)
EVQLVESGGGLVKPGGSLKLSCAASGYTFTSYVMHWVRQAPGKGLEWIGY
INPYNDGTKYNEKFQGRVTISSDKSISTAYMELSSLRSEDTAMYYCARGT
YYYGTRVFDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPDVFLFPPK
PKDTLMISRTPEVTCVVVDVSHEDPEVQFNWYVDGVEVHNAKTKPREEQF
NSTFRVVSVLTVVHQDWLNGKEYKCKVSNKALPAPEEKTISKTKGQPREP
QVYTLPPSREEMTKNQVSLTCLVKGFYPSDIAVEWESNGQPENNYKTTPP
MLDSDGSFFLYSKLTVDKSRWQQGNVFSCSVMHEALHNHYTQKSLSLSPG
K

and a light chain having the sequence

(SEQ ID NO: 12)
DIVMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC.

In an embodiment the anti-CD19 antibody or antibody fragment thereof for use in the treatment of hematological cancer patients in combination with a polypeptide that blocks the SIRPα-CD47 innate immune checkpoint comprises a variable heavy chain of the sequence

(SEQ ID NO: 7)
EVQLVESGGGLVKPGGSLKLSCAASGYTFTSYVMHWVRQAPGKGLEWIGY
INPYNDGTKYNEKFQGRVTISSDKSISTAYMELSSLRSEDTAMYYCARGT
YYYGTRVFDYWGQGTLVTVSS

and a variable light chain of the sequence

(SEQ ID NO: 8)
DIVMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIK,

or a variable heavy chain and and a variable light chain that has at least 80%, at least 90%, at least 95%, at least 96%, at least 97%, at least 98% or at least 99% identity to the variable heavy chain of SEQ ID NO: 7 and to the variable light chain of SEQ ID NO: 8.

In an embodiment the anti-CD19 antibody or antibody fragment thereof for use in the treatment of hematological cancer patients in combination with a polypeptide that blocks the SIRPα-CD47 innate immune checkpoint comprises a variable heavy chain of the sequence

(SEQ ID NO: 7)
EVQLVESGGGLVKPGGSLKLSCAASGYTFTSYVMHWVRQAPGKGLEWIGY
INPYNDGTKYNEKFQGRVTISSDKSISTAYMELSSLRSEDTAMYYCARGT
YYYGTRVFDYWGQGTLVTVSS

and a variable light chain of the sequence

(SEQ ID NO: 8)
DIVMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIK,

or a variable heavy chain and and a variable light chain that has at least 80%, at least 90%, at least 95%, at least 96%, at least 97%, at least 98% or at least 99% identity to the variable heavy chain of SEQ ID NO: 7 and to the variable light chain of SEQ ID NO: 8, wherein the anti-CD19 antibody comprises an HCDR1 region comprising the sequence SYVMH (SEQ ID NO: 1), an HCDR2 region comprising the sequence NPYNDG (SEQ ID NO: 2), an HCDR3 region comprising the sequence GTYYYGTRVFDY (SEQ ID NO: 3), an LCDR1 region comprising the sequence RSSKSLQNVNGNTYLY (SEQ ID NO: 4), an LCDR2 region comprising the sequence RMSNLNS (SEQ ID NO: 5), and an LCDR3 region comprising the sequence MQHLEYPIT (SEQ ID NO: 6). In another embodiment the heavy chain region of the anti-CD19 antibody comprises amino acids 239D and 332E, wherein the Fc numbering is according to the EU index as in Kabat.

In a further embodiment the anti-CD19 antibody or antibody fragment thereof for use in the treatment of hematological cancer patients in combination with a polypeptide that blocks the SIRPα-CD47 innate immune checkpoint comprises a heavy chain having the sequence

(SEQ ID NO: 11)
EVQLVESGGGLVKPGGSLKLSCAASGYTFTSYVMHWVRQAPGKGLEWIGY
INPYNDGTKYNEKFQGRVTISSDKSISTAYMELSSLRSEDTAMYYCARGT
YYYGTRVFDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPDVFLFPPK
PKDTLMISRTPEVTCVVVDVSHEDPEVQFNWYVDGVEVHNAKTKPREEQF
NSTFRVVSVLTVVHQDWLNGKEYKCKVSNKALPAPEEKTISKTKGQPREP
QVYTLPPSREEMTKNQVSLTCLVKGFYPSDIAVEWESNGQPENNYKTTPP
MLDSDGSFFLYSKLTVDKSRWQQGNVFSCSVMHEALHNHYTQKSLSLSPG
K

and a light chain having the sequence

(SEQ ID NO: 12)
DIVMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC,

or a heavy chain and and a light chain that has at least 80%, at least 90%, at least 95%, at least 96%, at least 97%, at least 98% or at least 99% identity to the heavy chain of SEQ ID NO: 7 and to the light chain of SEQ ID NO: 8.

In a further embodiment the anti-CD19 antibody or antibody fragment thereof for use in the treatment of hematological cancer patients in combination with a polypeptide that blocks the SIRPα-CD47 innate immune checkpoint comprises a heavy chain having the sequence

(SEQ ID NO: 11)
EVQLVESGGGLVKPGGSLKLSCAASGYTFTSYVMHWVRQAPGKGLEWIGY
INPYNDGTKYNEKFQGRVTISSDKSISTAYMELSSLRSEDTAMYYCARGT
YYYGTRVFDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPDVFLFPPK
PKDTLMISRTPEVTCVVVDVSHEDPEVQFNWYVDGVEVHNAKTKPREEQF
NSTFRVVSVLTVVHQDWLNGKEYKCKVSNKALPAPEEKTISKTKGQPREP
QVYTLPPSREEMTKNQVSLTCLVKGFYPSDIAVEWESNGQPENNYKTTPP
MLDSDGSFFLYSKLTVDKSRWQQGNVFSCSVMHEALHNHYTQKSLSLSPG
K

and a light chain having the sequence

(SEQ ID NO: 12)
DIVMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC,

or a heavy chain and and a light chain that has at least 80%, at least 90%, at least 95%, at least 96%, at least 97%, at least 98% or at least 99% identity to the heavy chain of SEQ ID NO: 7 and to the light chain of SEQ ID NO: 8 and wherein the anti-CD19 antibody comprises an HCDR1 region comprising the sequence SYVMH (SEQ ID NO: 1), an HCDR2 region comprising the sequence NPYNDG (SEQ ID NO: 2), an HCDR3 region comprising the sequence GTYYYGTRVFDY (SEQ ID NO: 3), an LCDR1 region comprising the sequence RSSKSLQNVNGNTYLY (SEQ ID NO: 4), an LCDR2 region comprising the sequence RMSNLNS (SEQ ID NO: 5), and an LCDR3 region comprising the sequence MQHLEYPIT (SEQ ID NO: 6). In another embodiment the heavy chain region of the anti-CD19 antibody comprises amino acids 239D and 332E, wherein the Fc numbering is according to the EU index as in Kabat.

In one embodiment the present disclosure provides an anti-CD19 antibody or antibody fragment thereof wherein said anti-CD19 antibody or antibody fragment thereof is administered in a concentration of 12 mg/kg.

In a further embodiment, the anti-CD19 antibody or antibody fragment thereof is administered weekly, bi-weekly or monthly. In a further embodiment the anti-CD19 antibody or antibody fragment thereof is administered weekly for the first 3 months and bi-weekly for at least the next 3 months. In a further embodiment, the anti-CD19 antibody or antibody fragment thereof is administered weekly for the first 3 months. In a further embodiment the anti-CD19 antibody or antibody fragment thereof is administered weekly for the first 3 months and bi-weekly for at least the next 3 months. In another embodiment the anti-CD19 antibody or antibody fragment thereof is administered weekly for the first 3 months, bi-weekly for the next 3 months and monthly thereafter. In yet another embodiment the anti-CD19 antibody or antibody fragment thereof is administered weekly for the first 3 months, bi-weekly for the next 3 months and monthly thereafter.

The present disclosure provides an antibody or antibody fragment specific for

CD19 for use in the treatment of cancer wherein said antibody or antibody fragment specific for CD19 is administered in combination with a polypeptide that blocks the SIRPα-CD47 innate immune checkpoint.

The present disclosure provides a pharmaceutical combination, comprising an antibody or antibody fragment specific for CD19 and a polypeptide that blocks the SIRPα-CD47 innate immune checkpoint for use in the treatment of cancer.

A therapeutically effective dose of the polypeptide that blocks the SIRPα-CD47 innate immune checkpoint (e.g. an anti-CD47 antibody or antibody fragment) can depend on the specific agent used, but is usually about 2 mg/kg body weight or more, about 4 mg/kg body weight or more, about 6 mg/kg body weight or more, about 8 mg/kg body weight or more, about 10 mg/kg body weight or more, about 12 mg/kg body weight or more, about 14 mg/kg body weight or more, about 16 mg/kg body weight or more, about 18 mg/kg body weight or more about 20 mg/kg body weight or more, about 25 mg/kg or more, about 30 mg/kg or more, about 35 mg/kg or more, about 40 mg/kg or more, about 45 mg/kg or more, about 50 mg/kg or more, or about 55 mg/kg or more, or about 60 mg/kg or more, or about 65 mg/kg or more, or about 70 mg/kg or more.

In some embodiments, the therapeutically effective dose of the polypeptide that blocks the SIRPα-CD47 innate immune checkpoint is 2, 4, 5, 6, 7, 8, 9, 10, 11, 12, 13, 14, 15, 16, 17, 18, 19, 20, 30, 45, 60, or 70 mg/kg. In some embodiments, the therapeutically effective dose of the polypeptide that blocks the SIRPα-CD47 innate immune checkpoint is 20 to 60 mg/kg.

The dose needed to achieve and/or maintain a particular serum level of the administered composition is proportional to the amount of time between doses and inversely proportional to the number of doses administered. Thus, as the frequency of dosing increases, the needed dose decreases. The optimization of dosing strategies will be readily understood and practiced by one of ordinary skill in the art. An exemplary treatment regime entails administration once every two weeks or once a month or once every 3 to 6 months. Therapeutic entities of the present invention are usually administered on multiple occasions.

Intervals between single dosages can be weekly, monthly or yearly. Intervals can also be irregular as indicated by measuring blood levels of the therapeutic entity in the patient. Alternatively, therapeutic entities of the present invention can be administered as a sustained release formulation, in which case less frequent administration is used. Dosage and frequency vary depending on the half-life of the polypeptide in the patient.

A maintenance dose is a dose intended to be a therapeutically effective dose. For example, in experiments to determine the therapeutically effective dose, multiple different maintenance doses may be administered to different subjects. As such, some of the maintenance doses may be therapeutically effective doses and others may be sub-therapeutic doses.

In still other embodiments, methods of the present invention include treating, reducing or preventing tumor growth, tumor metastasis or tumor invasion of cancers including carcinomas, hematologic cancers, melanomas, sarcomas, gliomas, etc. For prophylactic applications, pharmaceutical compositions or medicaments are administered to a patient susceptible to, or otherwise at risk of disease in an amount sufficient to eliminate or reduce the risk, lessen the severity, or delay the outset of the disease, including biochemical, histologic and/or behavioral symptoms of the disease, its complications and intermediate pathological phenotypes presenting during development of the disease.

Toxicity of the combined agents described herein can be determined by standard pharmaceutical procedures in cell cultures or experimental animals, e.g., by determining the LD50 (the dose lethal to 50% of the population) or the LD100 (the dose lethal to 100% of the population). The dose ratio between toxic and therapeutic effect is the therapeutic index. The data obtained from these cell culture assays and animal studies can be used in formulating a dosage range that is not toxic for use in human. The dosage of the proteins described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition.

Effective doses of the combined agents of the present invention for the treatment of cancer vary depending upon many different factors, including means of administration, target site, physiological state of the patient, whether the patient is human or an animal, other medications administered, and whether treatment is prophylactic or therapeutic. Usually, the patient is a human, but nonhuman mammals may also be treated, e.g. companion animals such as dogs, cats, horses, etc., laboratory mammals such as rabbits, mice, rats, etc., and the like. Treatment dosages can be titrated to optimize safety and efficacy.

The present disclosure provides a pharmaceutical combination, comprising an antibody or antibody fragment specific for CD19 and a polypeptide that blocks the SIRPα-CD47 innate immune checkpoint for use in the treatment of cancer.

The present disclosure provides an antibody or antibody fragment specific for CD19 for use in the treatment of cancer wherein said antibody or antibody fragment specific for CD19 is administered in combination with a polypeptide that blocks the SIRPα-CD47 innate immune checkpoint wherein the step of administering is carried out by administering the antibody specific for CD19 and the polypeptide that blocks the SIRPα-CD47 innate immune checkpoint in combination in a simultaneous, in order, or in reverse-order manner.

In another embodiment the present disclosure provides the use of a pharmaceutical combination comprising an antibody or antibody fragment specific for CD19 and a polypeptide that blocks the SIRPα-CD47 innate immune checkpoint for preparing a medicament for treatment of cancer. In another embodiment the present disclosure provides the use of a pharmaceutical combination comprising an antibody or antibody fragment specific for CD19 and a polypeptide that blocks the SIRPα-CD47 innate immune checkpoint for the preparation of a medicament for treatment of cancer.

In another embodiment the present disclosure provides a method for use in the treatment of cancer, comprising a step of administering, to a subject, an antibody specific for CD19 and a polypeptide that blocks the SIRPα-CD47 innate immune checkpoint in combination. In another embodiment the present disclosure provides a method for use in the treatment of cancer, comprising a step of administering, to a subject, an antibody specific for CD19 and a polypeptide that blocks the SIRPα-CD47 innate immune checkpoint in combination wherein the step of administering is carried out by administering the antibody specific for CD19 and the polypeptide that blocks the SIRPα-CD47 innate immune checkpoint in combination in a simultaneous, in order, or in reverse-order manner.

### Combinations

The present disclosure provides an anti-CD19 antibody or antibody fragment thereof in combination with an antibody or antibody fragment specific for CD19 and a polypeptide that blocks the SIRPα-CD47 innate immune checkpoint for use in the treatment of hematological cancer wherein said anti-CD19 antibody or antibody fragment thereof and an antibody or antibody fragment specific for CD19 and the polypeptide that blocks the SIRPα-CD47 innate immune checkpoint are administered in combination with one or more pharmaceutical agents. In one embodiment of the present disclosure said anti-CD19 antibody or antibody fragment thereof and an antibody or antibody fragment specific for CD19 and a polypeptide that blocks the SIRPα-CD47 innate immune checkpoint are administered in combination with a pharmaceutical agent. In another embodiment of the present disclosure said anti-CD19 antibody or antibody fragment thereof and said polypeptide that blocks the SIRPα-CD47 innate immune checkpoint are administered in combination with one or more additional pharmaceutical agents. In one aspect said pharmaceutical agent is an additional pharmaceutical agent. In one embodiment of the present disclosure said pharmaceutical agent is a biologic or a chemotherapeutic agent. In another embodiment of the present disclosure said pharmaceutical agent is a therapeutic antibody or antibody fragment, a nitrogen mustard, a purine analog, a thalidomide analog, a phosphoinositide 3-kinase inhibitor, a BCL-2 inhibitor or a bruton's tyrosine kinase (BTK) inhibitor. In a further embodiment said pharmaceutical agent is rituximab, R-CHOP, cyclophosphamide, chlorambucil, uramustine, ifosfamide, melphalan, bendamustine, mercaptopurine, azathioprine, thioguanine, fludarabine, thalidomide, lenalidomide, pomalidomide, idelalisib, duvelisib, copanlisib, ibrutinib or venetoclax.

In another embodiment the present disclosure provides an anti-CD19 antibody or antibody fragment thereof and a polypeptide that blocks the SIRPα-CD47 innate immune checkpoint for use in the treatment of hematological cancer wherein said anti-CD19 antibody or antibody fragment thereof and polypeptide that blocks the SIRPα-CD47 innate immune checkpoint are administered in combination with rituximab, R-CHOP, cyclophosphamide, chlorambucil, uramustine, ifosfamide, melphalan, bendamustine, mercaptopurine, azathioprine, thioguanine, fludarabine, thalidomide, lenalidomide, pomalidomide, idelalisib, duvelisib, copanlisib, ibrutinib or venetoclax.

In one aspect the present disclosure provides a pharmaceutical combination comprising an anti-CD19 antibody or antibody fragment thereof and a polypeptide that blocks the SIRPα-CD47 innate immune checkpoint for use in the treatment of cancer, wherein said pharmaceutical combination has a synergistic effect.

In some embodiments, said synergistic effect is improved overall survival (OS), extended progression free survival (PFS), increased response rate (RR) or increased or enhanced cancer cell clearance.

In some embodiments said synergistic effect is increased cancer cell death, decreased cancer cell growth or increased killing of non-Hodgkin's lymphoma cells. In some other embodiments such non-Hodgkin's lymphoma cells are cell lines, derived from Diffuse Large B Cell Lymphoma (DBLCL), Burkitt Lymphoma or Mantle Cell Lymphoma (MCL). In some other embodiments such non-Hodgkin's lymphoma cells are Raji, RCK8, Toledo, U2932, CA46, JVM-2, Ramos, Daudi or SU-DHL-6 cells.

In some embodiments said synergistic effect is increased survival, reduced tumor volume, or reduced tumor growth in a lymphoma mouse model. In some other embodiments such lymphoma mouse model is a xenograft model using cells derived from Diffuse Large B Cell Lymphoma (DBLCL), Burkitt Lymphoma or Mantle Cell Lymphoma (MCL). . In some other embodiments such lymphoma mouse model is a xenograft model using are Raji, RCK8, Toledo, U2932, CA46, JVM-2, Ramos, Daudi or SU-DHL-6 cells.

In another embodiment the pharmaceutical combination comprising an anti-CD19 antibody or antibody fragment thereof and a polypeptide that blocks the SIRPα-CD47 innate immune checkpoint for use in the treatment of cancer is a synergistic combination.

In one aspect the present disclosure provides a pharmaceutical combination comprising an anti-CD19 antibody or antibody fragment thereof and an anti-CD47 antibody or antibody fragment thereof for use in the treatment of hematological cancer, wherein the anti-CD19 antibody or antibody fragment thereof comprises a heavy chain variable region of

(SEQ ID NO: 7)
EVQLVESGGGLVKPGGSLKLSCAASGYTFTSYVMHWVRQAPGKGLEWIGY
INPYNDGTKYNEKFQGRVTISSDKSISTAYMELSSLRSEDTAMYYCARGT
YYYGTRVFDYWGQGTLVTVSS
and a light chain variable region of
(SEQ ID NO: 8)
DIVMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIK

and wherein the anti-CD47 antibody or fragment thereof comprises a heavy chain variable region of

(SEQ ID NO: 30)
QVQLVQSGAEVKKPGASVKVSCKASGYTFTNYNMHWVRQAPGQRLEWMGT
IYPGNDDTSYNQKFKDRVTITADTSASTAYMELSSLRSEDTAVYYCARGG
YRAMDYWGQGTLVTVSS
and a light chain variable region of
(SEQ ID NO: 31)
DIVMTQSPLSLPVTPGEPASISCRSSQSIVYSNGNTYLGWYLQKPGQSPQ
LLIYKVSNRFSGVPDRFSGSGSGTDFTLKISRVEAEDVGVYYCFQGSHVP
YTFGQGTKLEIK

wherein said pharmaceutical combination has a synergistic effect. In one embodiment the hematological cancer is chronic lymphocytic leukemia (CLL), non-Hodgkin's lymphoma (NHL), small lymphocytic lymphoma (SLL) or acute lymphoblastic leukemia (ALL). In another embodiment said pharmaceutical combination comprising said anti-CD19 antibody or antibody fragment thereof and said anti-CD47 antibody or antibody fragment thereof is a synergistic combination. In another embodiment the hematological cancer is non-Hodgkin's lymphoma (NHL). In a further embodiment the non-Hodgkin's lymphoma is selected from the group consisting of follicular lymphoma, small lymphocytic lymphoma, mucosa-associated lymphoid tissue, marginal zone lymphoma, diffuse large B cell lymphoma, Burkitt's lymphoma and mantle cell lymphoma. In another embodiment the hematological cancer is diffuse large B cell lymphoma.

In one aspect the present disclosure provides a pharmaceutical combination comprising an anti-CD19 antibody or antibody fragment thereof and an anti-CD47 antibody or antibody fragment thereof for use in the treatment of hematological cancer, wherein the anti-CD19 antibody or antibody fragment thereof comprises a heavy chain region of

(SEQ ID NO: 11)
EVQLVESGGGLVKPGGSLKLSCAASGYTFTSYVMHWVRQAPGKGLEWIGY
INPYNDGTKYNEKFQGRVTISSDKSISTAYMELSSLRSEDTAMYYCARGT
YYYGTRVFDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPDVFLFPPK
PKDTLMISRTPEVTCVVVDVSHEDPEVQFNWYVDGVEVHNAKTKPREEQF
NSTFRVVSVLTVVHQDWLNGKEYKCKVSNKALPAPEEKTISKTKGQPREP
QVYTLPPSREEMTKNQVSLTCLVKGFYPSDIAVEWESNGQPENNYKTTPP
MLDSDGSFFLYSKLTVDKSRWQQGNVFSCSVMHEALHNHYTQKSLSLSPG
K

and a light chain region of

(SEQ ID NO: 12)
DIVMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC

and wherein the anti-CD47 antibody or fragment thereof comprises a heavy chain of

(SEQ ID NO: 34)
QVQLVQSGAEVKKPGASVKVSCKASGYTFTNYNMHWVRQAPGQRLEWMGT
IYPGNDDTSYNQKFKDRVTITADTSASTAYMELSSLRSEDTAVYYCARGG
YRAMDYWGQGTLVTVSSASTKGPSVFPLAPCSRSTSESTAALGCLVKDYF
PEPVTVSWNSGALTSGVHTFPAVLQSSGLYSLSSVVTVPSSSLGTKTYTC
NVDHKPSNTKVDKRVESKYGPPCPPCPAPEFLGGPSVFLFPPKPKDTLMI
SRTPEVTCVVVDVSQEDPEVQFNWYVDGVEVHNAKTKPREEQFNSTYRVV
SVLTVLHQDWLNGKEYKCKVSNKGLPSSIEKTISKAKGQPREPQVYTLPP
SQEEMTKNQVSLTCLVKGFYPSDIAVEWESNGQPENNYKTTPPVLDSDGS
FFLYSRLTVDKSRWQEGNVFSCSVMHEALHNHYTQKSLSLSLGK

and a light chain

(SEQ ID NO: 35)
DIVMTQSPLSLPVTPGEPASISCRSSQSIVYSNGNTYLGWYLQKPGQSPQ
LLIYKVSNRFSGVPDRFSGSGSGTDFTLKISRVEAEDVGVYYCFQGSHVP
YTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC

and wherein said pharmaceutical combination has a synergistic effect. In another embodiment said pharmaceutical combination comprising said anti-CD19 antibody or antibody fragment thereof and said anti-CD47 antibody or antibody fragment thereof is a synergistic combination. In one embodiment the hematological cancer is chronic lymphocytic leukemia (CLL), non-Hodgkin's lymphoma (NHL), small lymphocytic lymphoma (SLL) or acute lymphoblastic leukemia (ALL). In another embodiment the hematological cancer is non-Hodgkin's lymphoma (NHL). In a further embodiment the non-Hodgkin's lymphoma is selected from the group consisting of follicular lymphoma, small lymphocytic lymphoma, mucosa-associated lymphoid tissue, marginal zone lymphoma, diffuse large B cell lymphoma, Burkitt's lymphoma and mantle cell lymphoma. In another embodiment the hematological cancer is diffuse large B cell lymphoma.

### Antibody Sequences

TABLE 1
Tafasitamab (MOR208)

| | SEQ ID NO: | Amino Acids |
|---|---|---|
| HCDR1 | SEQ ID NO: 1 | SYVMH |
| HCDR2 | SEQ ID NO: 2 | NPYNDG |
| HCDR3 | SEQ ID NO: 3 | GTYYYGTRVFDY |
| LCDR1 | SEQ ID NO: 4 | RSSKSLQNVNGNTYLY |
| LCDR2 | SEQ ID NO: 5 | RMSNLNS |
| LCDR3 | SEQ ID NO: 6 | MQHLEYPIT |
| VH | SEQ ID NO: 7 | EVQLVESGGGLVKPGGSLKLSCAASGYTFTSY |
| | | VMHWVRQAPGKGLEWIGYINPYNDGTKYNEK |
| | | FQGRVTISSDKSISTAYMELSSLRSEDTAMYYC |
| | | ARGTYYYGTRVFDYWGQGTLVTVSS |
| VL | SEQ ID NO: 8 | DIVMTQSPATLSLSPGERATLSCRSSKSLQNV |
| | | NGNTYLYWFQQKPGQSPQLLIYRMSNLNSGV |
| | | PDRFSGSGSGTEFTLTISSLEPEDFAVYYCMQ |
| | | HLEYPITFGAGTKLEIK |
| Heavy chain constant | SEQ ID NO: 9 | ASTKGPSVFPLAPSSKSTSGGTAALGCLVKDY |
| domain | | FPEPVTVSWNSGALTSGVHTFPAVLQSSGLYS |
| | | LSSVVTVPSSSLGTQTYICNVNHKPSNTKVDK |
| | | KVEPKSCDKTHTCPPCPAPELLGGPDVFLFPP |
| | | KPKDTLMISRTPEVTCVVVDVSHEDPEVQFNW |
| | | YVDGVEVHNAKTKPREEQFNSTFRVVSVLTVV |
| | | HQDWLNGKEYKCKVSNKALPAPEEKTISKTKG |
| | | QPREPQVYTLPPSREEMTKNQVSLTCLVKGFY |
| | | PSDIAVEWESNGQPENNYKTTPPMLDSDGSF |
| | | FLYSKLTVDKSRWQQGNVFSCSVMHEALHNH |
| | | YTQKSLSLSPGK |
| Light chain constant | SEQ ID NO: 10 | RTVAAPSVFIFPPSDEQLKSGTASWCLLNNFY |
| domain | | PREAKVQWKVDNALQSGNSQESVTEQDSKD |
| | | STYSLSSTLTLSKADYEKHKVYACEVTHQGLS |
| | | SPVTKSFNRGEC |
| Full Heavy chain | SEQ ID NO: 11 | EVQLVESGGGLVKPGGSLKLSCAASGYTFTSY |
| | | VMHWVRQAPGKGLEWIGYINPYNDGTKYNEK |
| | | FQGRVTISSDKSISTAYMELSSLRSEDTAMYYC |
| | | ARGTYYYGTRVFDYWGQGTLVTVSSASTKGP |
| | | SVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKS |
| | | CDKTHTCPPCPAPELLGGPDVFLFPPKPKDTL |
| | | MISRTPEVTCVVVDVSHEDPEVQFNWYVDGV |
| | | EVHNAKTKPREEQFNSTFRVVSVLTVVHQDW |
| | | LNGKEYKCKVSNKALPAPEEKTISKTKGQPRE |
| | | PQVYTLPPSREEMTKNQVSLTCLVKGFYPSDI |
| | | AVEWESNGQPENNYKTTPPMLDSDGSFFLYS |
| | | KLTVDKSRWQQGNVFSCSVMHEALHNHYTQK |
| | | SLSLSPGK |
| Full Light chain | SEQ ID NO: 12 | DIVMTQSPATLSLSPGERATLSCRSSKSLQNV |
| | | NGNTYLYWFQQKPGQSPQLLIYRMSNLNSGV |
| | | PDRFSGSGSGTEFTLTISSLEPEDFAVYYCMQ |
| | | HLEYPITFGAGTKLEIKRTVAAPSVFIFPPSDEQ |
| | | LKSGTASVVCLLNNFYPREAKVQWKVDNALQ |
| | | SGNSQESVTEQDSKDSTYSLSSTLTLSKADYE |
| | | KHKVYACEVTHQGLSSPVTKSFNRGEC |

TABLE 2
B6H12

| | SEQ ID NO: | Amino Acids |
|---|---|---|
| HCDR1 | SEQ ID NO: 14 | GYGMS |
| HCDR2 | SEQ ID NO: 15 | TITSGGTYTYYPDSVKG |
| HCDR3 | SEQ ID NO: 16 | SLAGNAMDY |
| LCDR1 | SEQ ID NO: 17 | RASQTISD |
| LCDR2 | SEQ ID NO: 18 | FASQSIS |
| LCDR3 | SEQ ID NO: 19 | QNGHGFPRT |
| VH | SEQ ID NO: 20 | EVQLVESGGDLVKPGGSLKLSCAASGF |
| | | TFSGYGMSWVRQTPDKRLEWVATITSG |
| | | GTYTYYPDSVKGRFTISRDNAKNTLYL |
| | | QIDSLKSEDTAIYFCARSLAGNAMDYW |
| | | GQGTSVTVSS |
| VL | SEQ ID NO: 21 | DIVMTQSPATLSVTPGDRVSLSCRASQ |
| | | TISDYLHWYQQKSHESPRLLIKFASQS |
| | | ISGIPSRFSGSGSGSDFTLSINSVEPE |
| | | DVGVYYCQNGHGFPRTFGGGTKLEIK |

TABLE 3
5F9 and variants

| | SEQ ID NO: | Amino Acids |
|---|---|---|
| HCDR1 | SEQ ID NO: 22 | NYNMH |
| HCDR2 | SEQ ID NO: 23 | TIYPGNDDTSYNQKFKD |
| HCDR3 | SEQ ID NO: 24 | GGYRAMDY |
| LCDR1 | SEQ ID NO: 25 | RSSQSIVYSNGNTYLG |
| LCDR2 | SEQ ID NO: 26 | KVSNRFS |
| LCDR3 | SEQ ID NO: 27 | FQGSHVPYT |
| VH | SEQ ID NO: 28 | QVQLVQSGAEVKKPGASVKVSCKASGYTFTN |
| | | YNMHWVRQAPGQGLEWIGTIYPGNDDTSYNQ |
| | | KFKDKATLTADKSTSTAYMELSSLRSEDTAVY |
| | | YCARGGYRAMDYWGQGTLVTVSS |
| VL | SEQ ID NO: 29 | DVVMTQSPLSLPVTPGEPASISCRSSQSIVYSN |
| | | GNTYLGWYLQKPGQSPKLLIYKVSNRFSGVPD |
| | | RFSGSGSGTDFTLKISRVEAEDVGVYHCFQGS |
| | | HVPYTFGGGTKVEIK |
| VH | SEQ ID NO: 30 | QVQLVQSGAEVKKPGASVKVSCKASGYTFTN |
| | | YNMHWVRQAPGQRLEWMGTIYPGNDDTSYN |
| | | QKFKDRVTITADTSASTAYMELSSLRSEDTAVY |
| | | YCARGGYRAMDYWGQGTLVTVSS |
| VL | SEQ ID NO: 31 | DIVMTQSPLSLPVTPGEPASISCRSSQSIVYSN |
| | | GNTYLGWYLQKPGQSPQLLIYKVSNRFSGVP |
| | | DRFSGSGSGTDFTLKISRVEAEDVGVYYCFQG |
| | | SHVPYTFGQGTKLEIK |
| VH | SEQ ID NO: 32 | QVQLVQSGAEVKKPGASVKVSCKASGYTFTN |
| | | YNMHWVRQAPGQRLEWIGTIYPGNDDTSYNQ |
| | | KFKDRATLTADKSASTAYMELSSLRSEDTAVY |
| | | YCARGGYRAMDYWGQGTLVTVSS |
| VL | SEQ ID NO: 33 | DVVMTQSPLSLPVTPGEPASISCRSSQSIVYSN |
| | | GNTYLGWYLQKPGQSPQLLIYKVSNRFSGVP |
| | | DRFSGSGSGTDFTLKISRVEAEDVGVYHCFQG |
| | | SHVPYTFGQGTKLEIK |
| Full Heavy | SEQ ID NO: 34 | QVQLVQSGAEVKKPGASVKVSCKASGYTFTN |
| chain | | YNMHWVRQAPGQRLEWMGTIYPGNDDTSYN |
| | | QKFKDRVTITADTSASTAYMELSSLRSEDTAVY |
| | | YCARGGYRAMDYWGQGTLVTVSSASTKGPS |
| | | VFPLAPCSRSTSESTAALGCLVKDYFPEPVTV |
| | | SWNSGALTSGVHTFPAVLQSSGLYSLSSVVTV |
| | | PSSSLGTKTYTCNVDHKPSNTKVDKRVESKYG |
| | | PPCPPCPAPEFLGGPSVFLFPPKPKDTLMISRT |
| | | PEVTCVVVDVSQEDPEVQFNWYVDGVEVHNA |
| | | KTKPREEQFNSTYRVVSVLTVLHQDWLNGKE |
| | | YKCKVSNKGLPSSIEKTISKAKGQPREPQVYTL |
| | | PPSQEEMTKNQVSLTCLVKGFYPSDIAVEWES |
| | | NGQPENNYKTTPPVLDSDGSFFLYSRLTVDKS |
| | | RWQEGNVFSCSVMHEALHNHYTQKSLSLSLG |
| | | K |
| Full Light | SEQ ID NO: 35 | DIVMTQSPLSLPVTPGEPASISCRSSQSIVYSN |
| chain | | GNTYLGWYLQKPGQSPQLLIYKVSNRFSGVP |
| | | DRFSGSGSGTDFTLKISRVEAEDVGVYYCFQG |
| | | SHVPYTFGQGTKLEIKRTVAAPSVFIFPPSDEQ |
| | | LKSGTASWCLLNNFYPREAKVQWKVDNALQ |
| | | SGNSQESVTEQDSKDSTYSLSSTLTLSKADYE |
| | | KHKVYACEVTHQGLSSPVTKSFNRGEC |

### EXAMPLES

### Example 1

### Efficacy of Tafasitamab (Anti-CD19 mAb) in Combination with CD47/SIRPα Blocking Antibody in Vitro

MOR208 Mediated ADCP Activity in Combination with CD47/SIRPα Blockade

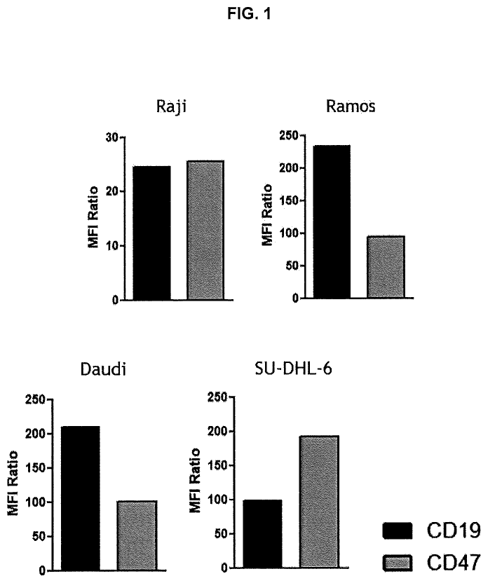

With in vitro assays it was tested, whether MOR208 (Tafasitamab) mediated phagocytosis can be further enhanced upon CD47/SIRPα blockade. Efficacy of Tafasitamab (anti-CD19 mAb) in combination with anti-CD47 (clone B6H12) functional antibody was determined in antibody-dependent cellular phagocytosis (ADCP) assays, where the THP-1 monocytic cell line or M1 and M2 macrophages served as effector cells. For this purpose the following cancer cell lines were characterized: Three Burkitt lymphoma cell lines (Raji, Ramos & Daudi) and one diffuse large B-cell lymphoma (DLBCL) cell line (SU-DHL-6). On these cancer cells CD19 and CD47 antigen expression levels were quantified (FIG. 1). Raji cells expressed the highest level of CD19, but also Ramos, Daudi and Toledo cells show high expression of CD19. The SU-DHL-6 cell line was the only cell line with low CD19 expression. It could be shown that CD47 is expressed on all analyzed cancer cell lines, with only SU-DHL-6 having low expression.

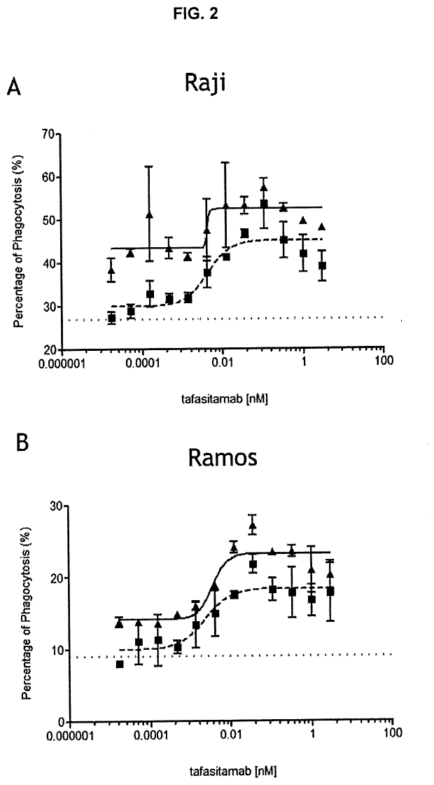

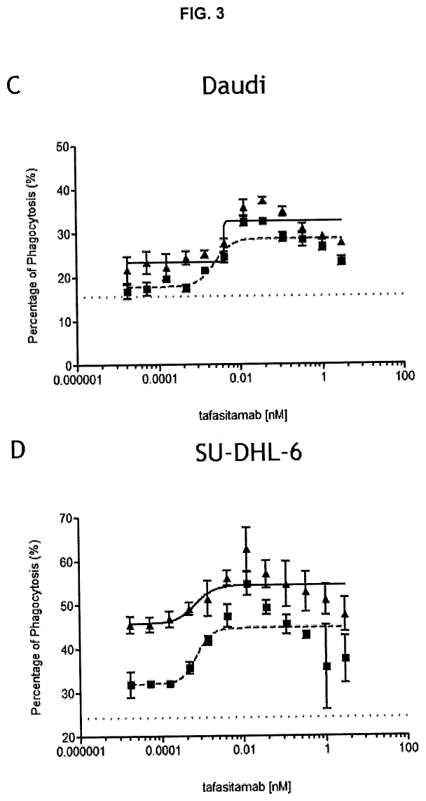

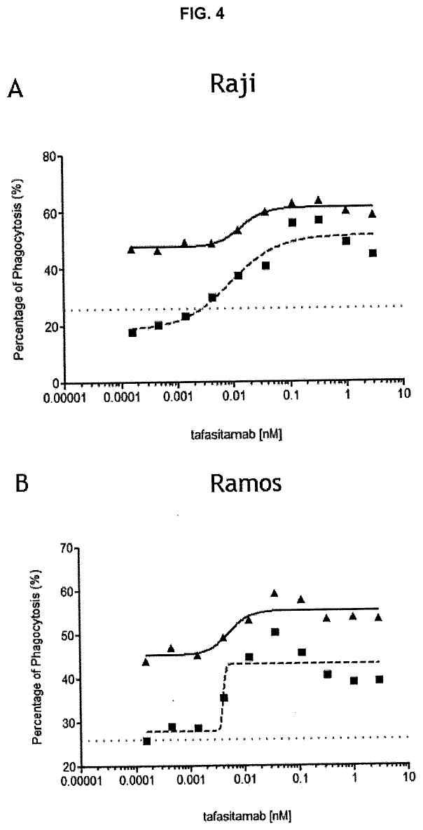

Ramos, Raji, Daudi and SU-DHL-6 cancer cell lines, were tested in ADCP assays with THP-1 monocytic cancer cells used as effector cells. Cancer cells were plated together with THP-1 cells in E:T (effector : target) ratio 1:2 and co-incubated in ADCP assay together with titration series of Tafasitamab combined with 3 nM anti-CD47 mAb (clone B6H12 (FIGS. 2 to 4). The benefit of anti-CD47 antibody on Tafasitamab mediated ADCP was assessed with a flow cytometry-based phagocytosis readout, where effector and target cells were stained with two different dyes (THP-1 cells with CFSE and cancer cells with Cell Trace™ Violet) In this way the obtained percentage of double positive cells represented the percentage of phagocytosis.

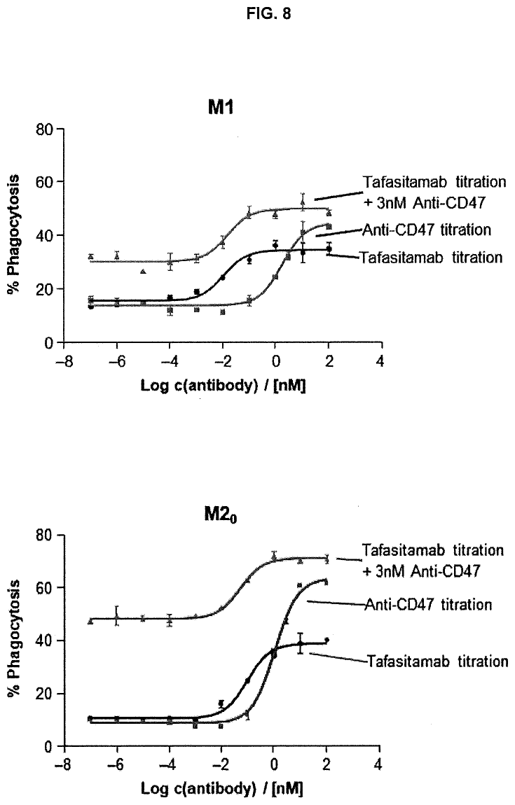

In addition, Ramos cancer cell lines, were tested in ADCP assays with M1 and M2 macrophages used as effector cells. For the generation of M1 and M2ₒ macrophages CD14+ monocytes were isolated from whole blood of healthy volunteers and matured into macrophages with 50 ng/mL M-CSF for 6 days. Macrophages were further polarized towards the M1 phenotype by addition of 10 ng/mL IFN-γ and 10 ng/mL LPS for 48 hours or treatment with 50 ng/mL M-CSF was continued to maintain the M2ₒ phenotype. Expression levels of macrophage phenotype markers CD80, CD86, CD163 and CD206 were analyzed and confirmed by flow cytometry. Ramos cells were plated together with M1 or M2ₒ macrophages in E:T (effector : target) ratio 1:2 and co-incubated together with titration series of Tafasitamab combined with 3 nM anti-CD47 mAb (clone B6H12). ADCP was analyzed by flow cytometry after 3 hours of treatment with MOR208 and anti-CD47 mAb (Clone B6H12) (FIG. 8).

### Results

ADCP assays showed that MOR208 mediated phagocytosis could be further enhanced upon combination with 3 nM of anti-CD47 mAb (clone B6H12) (FIGS. 2 to 4).

A comparable increase in phagocytosis of Ramos cells was observed for the combination of tafasitamab and CD47/SIRPα checkpoint blockade for both M1- as well as M2o-polarized macrophages (FIG. 8). The ADCP activity of M1- as well as M2ₒ-polarized macrophages was increased upon combining MOR208 with CD47/SIRPα checkpoint blockade in vitro. Overall the increase in phagocytotic activity driven by the combination was more pronounced for M2ₒ than for M1-polarized macrophages.

### Example 2

### Efficacy of Tafasitamab in Combination with Anti-CD47 Antibody in Vivo

For evaluating the combinational effect of an anti-CD47 antibody (clone B6H12) and Tafasitamab, three efficacy studies were performed with Ramos Burkitt lymphoma cells either in two subcutaneous (MOR208P015 and MOR208P016) and one disseminated survival tumor model (MOR208P014).

Because of the differential binding affinities (Kwong et al. 2014; Iwamoto et al. 2014) between human CD47 (hCD47) on Ramos cells and mouse SIRPα (mSIRPα) expressed on mouse macrophages and neutrophils, efficacy was tested in two different genetic mouse strains. In study MOR208P014 and MOR208P015 the Balb/c genetic background (SCID mice), described to closely mimic the human CD47-SIRPα checkpoint interaction, was used and in study MOR208P016 the NOD-SCID genetic strain was tested. NOD-SCID is most widely used in the literature (Chao et al. 2010a; Liu et al. 2015; Buatois et al. 2018; Kauder et al. 2018) for testing CD47 blocking compounds and reported to have a 10-fold higher hCD47 to mSIRPα checkpoint binding affinity, potentially leading to exaggerated anti-tumor effects (Huang et al. 2017).

The study readout for mono- and combinational efficacy was either the tumor volume (MOR208P015 and MOR208P016) or animal survival (MOR208P014).

### Methods: In Vivo Studies—Experimental Outline and Analysis

Five to eight-week-old, female, C.8-17 SCID (CB17/Icr-Prkdcscid/IcrIcoCrl; in study MOR208P014; MOR208P015), NOD-SCID (NOD.CB17-Prkdc^{scid}/J; in study MOR208P016) were purchased from individual vendors (MOR208P015 & MOR208P016: Charles River Laboratories; MOR208P014: Envigo). Animals were housed in IVC cages (type II, polysulphone cages), four-five per cage, in a 12-hour light/dark cycle and acclimated in the laboratories for one week prior to experimentation. All animals received filtered water and a special vehicle or test article containing nude mouse diet (placebo chow; Sniff, article no.: V1244-000).

### Cell Culture of Ramos Cells in All in Vivo Studies

Ramos human Burkitt lymphoma cells were cultured in RPMI 1640 supplemented with 20% fetal bovine serum, non-essential amino acids (2 mM L-glutamine) and sodium pyruvate in a suspension culture. The cells were serially passaged until a sufficient cell number was established for injection. Cells were counted and viability assessed using a 0.25% trypan blue exclusion assay, before and after cell subcutaneous inoculation into mice.

### MOR208P014—Efficacy Study in Ramos Disseminated Survival Model

### Tumor Cell Inoculation and Randomization

For proper orthotopic tumor cell take in SCID mice, animals were treated with 25 mg/kg Cyclophosphamide via intraperitoneal injection twice daily, 12 hours apart, starting two days prior to tumor cell inoculation. On the day of cell inoculation (Day 0), mice were weighed, randomised by body weight (based on Day 0 body weight measurements) into groups of fifteen and inoculated with 1×10⁶ RAMOS cells (in 100 μL) into the tail vein.

### Treatment and Assessment of Efficacy Parameters

Five days after cell inoculation antibody treatment commenced. Here, the CD47 antibody (clone B6H12; 4 mg/kg; BioXCell; Catalog#: BE0019-1; Lot#: 655117M2) was administered by intraperitoneal injection, three times a week. Tafasitamab (3 mg/kg) was administered intravenously twice a week and vehicle treated groups were injected with phosphate buffered saline intraperitoneally also twice a week. Treatments with described test articles were performed in total for three weeks.

Throughout the study animals were closely monitored for showing signs of morbidity, such as body weight loss, signs of pain and distress, appearance and behavior, which are all a clear causes for animal termination. The animal survival was further summarized in Kaplan and Maier graphs.

For statistical evaluations the log-rank (Mantel-Cox) test was used. All statistical analysis were done using GraphPad Prism. A p-value of less than 0.05 was considered significant.

### MOR208P015—Efficacy Study in Ramos-SCID Subcutaneous Tumor Model

### Tumor Cell Inoculation and Randomization

5×10⁶ Ramos tumor cells (in Cultrex basement membrane) were implanted subcutaneously in the right flank, using a 23 gauge ½ needle into C.8-17 SCID mice. The injection volume was 0.2 mL per mouse. The date of tumor implant was recorded as day 0. Once growing tumors reached a size of 70-150 mm³ animals were randomized in their respective treatment group and treatment commenced.

### Treatment and Assessment of Efficacy Parameters

In terms of antibody tretaments, Tafasitamab (10 mg/kg) was administered twice a week and the anti-CD47 (clone B6H12; 4 mg/kg; BioXCell; Catalog#: 6E0019-1; Lot#: 655117M2) three times a week. Vehicle treated groups were injected with phosphate buffered saline twice a week. All respective treatments were performed via intraperitoneal injection for up to four weeks.

Tumor size was measured twice weekly starting on day 0. The tumor volume was calculated using the equation for an ellipsoid sphere (I×w2)/2=mm3, where I and w refer to the larger and smaller dimensions collected at each measurement and assuming unit density. Changes in body weight were monitored initially daily at the first day of treatment and ending one day after the last treatment. Moribund animals, animals with excessive body weight loss (>25% of the body weight) or animals with a total tumor burden of 3,000 mm³ were terminated prior to the end of the study.

For statistical evaluation of the delayed tumor growth Kaplan and Meier graphs were generated, showing the time until a tumor volume of 3000 mm³ was reached. The log-rank Mantel-Cox test was used for evaluating statistical differences. All statistical analyses were done with GraphPad Prism. A p-value of less than 0.05 was considered significant.

### MOR208P016: Ramos Subcutaneous Model in NOD-SCID Mice

### Tumor Cell Inoculation and Randomization

1×10⁷ Ramos tumor cells were injected subcutaneously, in 200 μL of RPMI 1640 containing 50% (v/v) matrigel (ref. 356237, Corning), into the right flank by using a 23 gauge ½ needle into female C.B-17 SCID mice. Animals were randomized once tumors reached a mean volume of 100-200 mm³ and right after treatment with respective antibodies and vehicle control commenced.

### Treatment and Assessment of Efficacy Parameters

Tafasitamab (10 mg/kg, twice per week), anti-CD47 antibody (clone B6H12; 4 mg/kg; three times per week; BioXCell; Catalog#: BE0019-1; Lot#: 655117M2) and vehicle (Phosphate buffered saline) were administered intraperitoneally for up to four weeks.

Tumors were measured twice weekly starting on the day of tumor cell injection. Tumor volumes were calculated by using the equation for an ellipsoid sphere (I×w2)/2=mm³. Changes in body weight were monitored initially daily, staring at the first day and ending on the last day of treatment. Moribund animals, animals with excessive body weight loss (>25% of the body weight) or animals with a total tumor burden of 2,000 mm³ were terminated prior to the end of the study.

For statistical evaluation of the delayed tumor growth Kaplan and Meier graphs were generated, showing the time until a tumor volume of 1500 mm³ was reached. The log-rank Mantel-Cox test was used for evaluating statistical differences. All statistical analyses were done with GraphPad Prism. A p-value of less than 0.05 was considered significant.

### Results of the in Vivo Studies

### Efficacy of MOR208 & Anti-CD47 Antibody Combinations in an Disseminated Survival Model (MOR208P014)

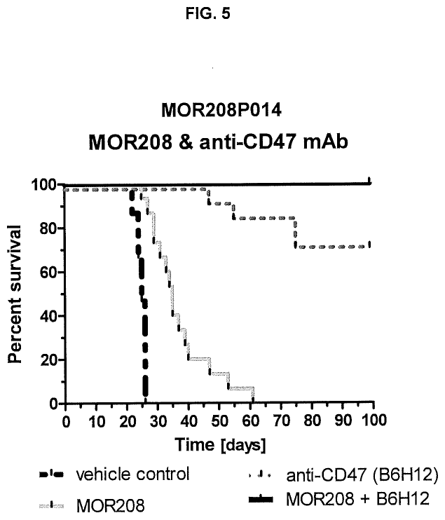

MOR208 treatment significantly improved median survival up to 40% compared to vehicle control (p<0.0001****). In addition the blockade of the CD47-SIRPα checkpoint with the anti-CD47 (clone B6H12) antibody in monotherapy, significantly improved animal survival up to 3 fold vs. vehicle control (p<0.0001****). Eleven out of fifteen animals remained on study until the end of in life phase. In terms of MOR208 & anti-CD47 combination this trend was even more pronounced. All fifteen animals remained alive until the end of the study (86H12 vs MOR208 & B6H12: p=0.0348*; MOR208 vs MOR208 & B6H12: p<0.0001****). No bio-statistical evaluations, concerning the power of this combinational effect could be performed, since all animals in the combinational treatment group remained on study. Data from in vivo study MOR208P014 is summarized in FIG. 5.

### MOR208 Combinational Efficacy in Ramos-SCID Subcutaneous Tumors (MOR208P015)

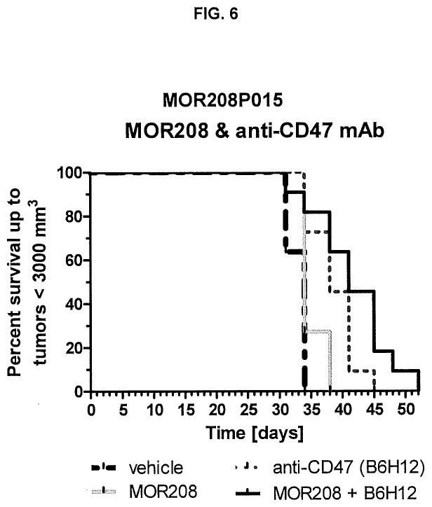

Evaluations of the delayed tumor growth were performed by using Kaplan-Meier curves, as described in the methods section. A minor but still significant delay in tumor growth was detected for MOR208 monotherapy, compared to vehicle control (vehicle vs. MOR208: p=0.0331*). In addition, the anti-CD47 mAb (clone B6H12) monotherapy showed a significant delayed tumor growth (vehicle vs. B6H12: p=0.0003***) of up to 12% compared to vehicle control. In terms of the MOR208 & anti-CD47 mAb combination the delay in tumor growth was even more pronounced. A 20% decrease in tumor load compared to MOR208 monotherapy and a 8% decrease compared to the anti-CD47 monotherapy was detected. But this effects is not significant, compared to the anti-CD47 mAb monotherapy control (p=0.0985). Data from in vivo study MOR208P015 is summarized in FIG. 6.

### MOR208 Combinational Efficacy in Ramos-NOD-SCID Subcutaneous Tumors (MOR208P016)

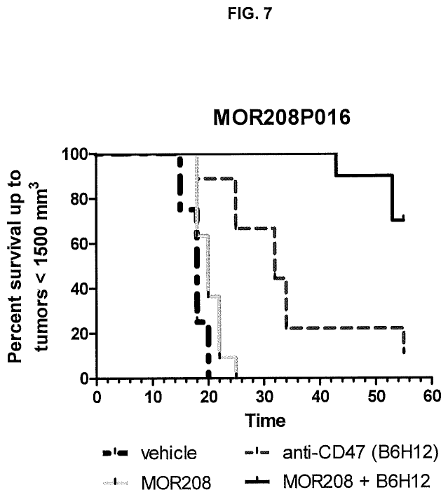

Similar to study MOR208P015, the delay in tumor growth is summarized by Kaplan-Meier curves. MOR208 mono-therapy significantly delayed tumor growth up to 11% compared to vehicle control (vehicle vs. MOR208: p=0.0095**). The anti-CD47 mAb (clone B6H12) single agent efficacy in this model was very pronounced and a 78% delay in tumor growth compared to vehicle control was observed (vehicle vs. B6H12: p <0.0001*****). However, the monotherapeutic efficacy more increased in combination with MOR208, with highly significant effects, compared to respective monotherapy controls (MOR208 vs. MOR208 &,B6H12 p<0.0001****; B6H12 vs. MOR208 & B6H12: p=0.0017**). All data from in vivo study MOR208P016 is summarized in FIG. 7.

### Example 3

### Efficacy of Tafasitamab in Combination with Magrolimab (Anti-CD47 Antibody)

This study was designed to evaluate whether the combination of an anti-CD19 antibody (tafasitamab) with magrolimab could increase the phagocytosis of B-cell lymphoma cells in vitro.

Six different cell lines, derived from either Diffuse Large B Cell Lymphoma (DBLCL), Burkitt Lymphoma, or Mantle Cell Lymphoma (MCL) were tested. Each cell line was fluorescently labeled with CellTrace CFSE dye, according to the manufacturer's indications. Human monocytes were isolated from leukocyte-enriched whole blood by incubation and subsequent purification with CD14-binding magnetic beads. The resulting monocytes were cultured in vitro in the presence of human recombinant macrophage colony stimulating factor (M-CSF) for 7-10 days, then harvested and counted prior to co-culture. Phagocytosis reactions were performed by co-culturing 50,000 human macrophages with 100,000 human cancer cells in a 100 ul volume in wells of a 96-well ultra-low attachment cell culture plate, along with magrolimab and/or tafasitamab treatments as indicated, at a final concentration of 10 ug/ml. Co-cultures were incubated at 37° C. for 2 hours, then transferred to ice to halt the reaction. Macrophages were stained using a labeled anti-CD11b antibody, and the reactions were analyzed on a flow cytometer. Phagocytic events were defined as CFSE+ CD11b+ events based on FMO controls. Such double positive events correspond to macrophages which have engulfed CFSE+ tumor cells. Phagocytosis was expressed as the fraction of macrophages positive for CFSE.

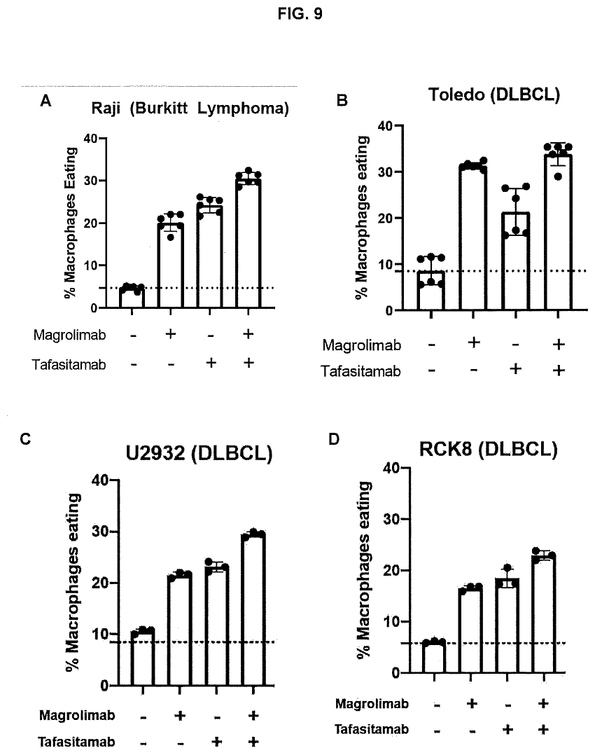

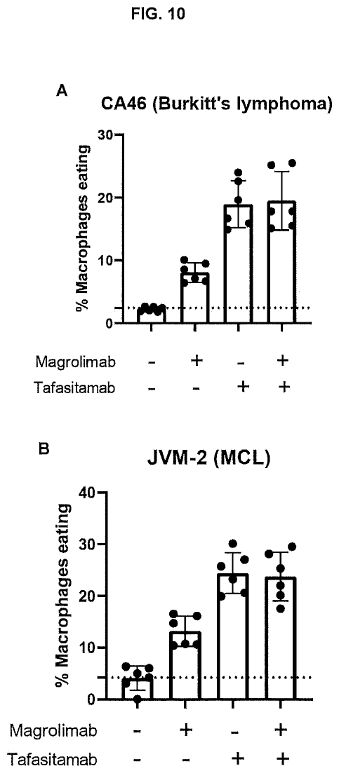

Of the cell lines tested, all six showed increased phagocytosis with magrolimab (anti-CD47) treatment alone. Similarly, all six showed increased phagocytosis with tafasitamab (anti-CD19) treatment alone. Of the six cell lines, four (Raji, RCK8, Toledo, and U2932) showed enhanced phagocytosis when treated with both magrolimab and tafasitamab, as compared to either single treatment (FIG. 9). Two of the six cell lines (CA46, JVM-2) did not show clearly enhanced efficacy of the combination when compared to tafasitamab alone (FIG. 10).

In summary, this study demonstrated that treatment with either magrolimab or tafasitamab can enhance in vitro phagocytosis of B-cell lymphomas; and that for a subset of B-cell lymphomas, the combination of the two drugs is more potent than either drug alone. These results are consistent with the conclusion that magrolimab and tafasitamab can show combinatorial efficacy when used in patients to treat B-cell lymphomas.

1-2. (canceled)

3. The method of claim 18, wherein the hematological cancer is chronic lymphocytic leukemia (CLL), non-Hodgkin's lymphoma (NHL), small lymphocytic lymphoma (SLL) or acute lymphoblastic leukemia (ALL).

4. The method of claim 3, wherein the NHL is selected from the group consisting of follicular lymphoma, small lymphocytic lymphoma, mucosa-associated lymphoid tissue, marginal zone lymphoma, diffuse large B cell lymphoma, Burkitt's lymphoma, and mantle cell lymphoma.

5. The method of claim 16, wherein said anti-CD19 antibody or antibody fragment thereof and the polypeptide that blocks the SIRPα-CD47 innate immune checkpoint are administered in a separate manner.

6. The method of claim 16, wherein said anti-CD19 antibody or antibody fragment thereof and the polypeptide that blocks the SIRPα-CD47 innate immune checkpoint are administered in a simultaneous manner.

7. The method of claim 16, wherein said anti-CD19 antibody or antibody fragment thereof comprises a heavy chain variable region comprising an HCDR1 region comprising the sequence SYVMH (SEQ ID NO: 1), an HCDR2 region comprising the sequence NPYNDG (SEQ ID NO: 2), and an HCDR3 region comprising the sequence GTYYYGTRVFDY (SEQ ID NO: 3) and a light chain variable region comprising an LCDR1 region comprising the sequence RSSKSLQNVNGNTYLY (SEQ ID NO: 4), an LCDR2 region comprising the sequence RMSNLNS (SEQ ID NO: 5), and an LCDR3 region comprising the sequence MQHLEYPIT (SEQ ID NO: 6).

8. The method of claim 16, wherein said anti-CD19 antibody or antibody fragment thereof comprises a heavy chain variable region of
(SEQ ID NO: 7)
EVQLVESGGGLVKPGGSLKLSCAASGYTFTSYVMHWVRQAPGKGLEWIGY
INPYNDGTKYNEKFQGRVTISSDKSISTAYMELSSLRSEDTAMYYCARGT
YYYGTRVFDYWGQGTLVTVSS
and a light chain variable region of
(SEQ ID NO: 8)
DIVMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIK.

9. The method of claim 16, wherein said anti-CD19 antibody comprises a heavy chain of
(SEQ ID NO: 11)
EVQLVESGGGLVKPGGSLKLSCAASGYTFTSYVMHWVRQAPGKGLEWIGY
INPYNDGTKYNEKFQGRVTISSDKSISTAYMELSSLRSEDTAMYYCARGT
YYYGTRVFDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPDVFLFPPK
PKDTLMISRTPEVTCVVVDVSHEDPEVQFNWYVDGVEVHNAKTKPREEQF
NSTFRVVSVLTVVHQDWLNGKEYKCKVSNKALPAPEEKTISKTKGQPREP
QVYTLPPSREEMTKNQVSLTCLVKGFYPSDIAVEWESNGQPENNYKTTPP
MLDSDGSFFLYSKLTVDKSRWQQGNVFSCSVMHEALHNHYTQKSLSLSPG
K

10. The method of claim 16, wherein said anti-CD19 antibody or antibody fragment thereof comprises a light chain of
(SEQ ID NO: 12)
DIVMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC.

11. The method of claim 16, wherein said polypeptide that blocks the SIRPα-CD47 innate immune checkpoint is a polypeptidic SIRPα reagent or an antibody or antibody fragment that specifically binds to human CD47 or human SIRPα.

12. The method of claim 16, wherein said polypeptide that blocks the SIRPα-CD47 innate immune checkpoint is an anti-CD47 antibody of fragment thereof comprising a heavy chain variable region comprising an HCDR1 region comprising the sequence NYNMH (SEQ ID NO: 22), an HCDR2 region comprising the sequence TIYPGNDDTSYNQKFKD (SEQ ID NO: 23), and an HCDR3 region comprising the sequence GGYRAMDY (SEQ ID NO: 24) and a light chain variable region comprising an LCDR1 region comprising the sequence RSSQSIVYSNGNTYLG (SEQ ID NO: 25), an LCDR2 region comprising the sequence KVSNRFS (SEQ ID NO: 26), and an LCDR3 region comprising the sequence FQGSHVPYT (SEQ ID NO: 27).

13. The method of claim 12, wherein said anti-CD47 antibody of fragment thereof comprises a heavy chain variable region of
(SEQ ID NO: 30)
QVQLVQSGAEVKKPGASVKVSCKASGYTFTNYNMHWVRQAPGQRLEWMGT
IYPGNDDTSYNQUKDRVTITADTSASTAYMELSSLRSEDTAVYYCARGGY
RAMDYWGQGTLVTVSS
and a light chain variable region of
(SEQ ID NO: 31)
DIVMTQSPLSLPVTPGEPASISCRSSQSIVYSNGNTYLGWYLQKPGQSPQ
LLIYKVSNRFSGVPDRFSGSGSGTDFTLKISRVEAEDVGVYYCFQGSHVP
YTFGQGTKLEIK.

14. The method of claim 13, wherein said anti-CD47 antibody of fragment thereof comprises a heavy chain of
(SEQ ID NO: 34)
QVQLVQSGAEVKKPGASVKVSCKASGYTFTNYNMHWVRQAPGQRLEWMGT
IYPGNDDTSYNQKFKDRVTITADTSASTAYMELSSLRSEDTAVYYCARGG
YRAMDYWGQGTLVTVSSASTKGPSVFPLAPCSRSTSESTAALGCLVKDYF
PEPVTVSWNSGALTSGVHTFPAVLQSSGLYSLSSVVTVPSSSLGTKTYTC
NVDHKPSNTKVDKRVESKYGPPCPPCPAPEFLGGPSVFLFPPKPKDTLMI
SRTPEVTCVVVDVSQEDPEVQFNWYVDGVEVHNAKTKPREEQFNSTYRVV
SVLTVLHQDWLNGKEYKCKVSNKGLPSSIEKTISKAKGQPREPQVYTLPP
SQEEMTKNQVSLTCLVKGFYPSDIAVEWESNGQPENNYKTTPPVLDSDGS
FFLYSRLTVDKSRWQEGNVFSCSVMHEALHNHYTQKSLSLSLGK
and a light chain of
(SEQ ID NO: 35)
DIVMTQSPLSLPVTPGEPASISCRSSQSIVYSNGNTYLGWYLQKPGQSPQ
LLIYKVSNRFSGVPDRFSGSGSGTDFTLKISRVEAEDVGVYYCFQGSHVP
YTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC

15. (canceled)

16. A method of treating a human subject having a cancer, comprising:
(a) administering to the human subject a polypeptide that blocks the SIRPα.-CD47 innate immune checkpoint; and
(b) administering to the human subject an anti-CD19 antibody or antibody fragment thereof.

17. A method of reducing the size of a cancer in a human subject, comprising:
(a) administering to the human subject a polypeptide that blocks the SIRPα-CD47 innate immune checkpoint; and
(b) administering to the human subject an anti-CD19 antibody or antibody fragment thereof.

18. The method according to claim 16, wherein the cancer is a hematological cancer.

19. The method according to claim 17, wherein the cancer is a hematological cancer.


SEQUENCE LISTING (35 sequences)

SEQ ID NO: 1 - protein, 5 aa, Artificial Sequence
  source: /note=“Description of Artificial Sequence Synthetic peptide”
SYVMH

SEQ ID NO: 2 - protein, 6 aa, Artificial Sequence
  source: /note=“Description of Artificial Sequence Synthetic peptide”
NPYNDG

SEQ ID NO: 3 - protein, 12 aa, Artificial Sequence
  source: /note=“Description of Artificial Sequence Synthetic peptide”
GTYYYGTRVFDY

SEQ ID NO: 4 - protein, 16 aa, Artificial Sequence
  source: /note=“Description of Artificial Sequence Synthetic peptide”
RSSKSLQNVNGNTYLY

SEQ ID NO: 5 - protein, 7 aa, Artificial Sequence
  source: /note=“Description of Artificial Sequence Synthetic peptide”
RMSNLNS

SEQ ID NO: 6 - protein, 9 aa, Artificial Sequence
  source: /note=“Description of Artificial Sequence Synthetic peptide”
MQHLEYPIT

SEQ ID NO: 7 - protein, 121 aa, Artificial Sequence
  source: /note=“Description of Artificial Sequence Synthetic polypeptide”
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSS

SEQ ID NO: 8 - protein, 112 aa, Artificial Sequence
  source: /note=“Description of Artificial Sequence Synthetic polypeptide”
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIK

SEQ ID NO: 9 - protein, 330 aa, Artificial Sequence
  source: /note=“Description of Artificial Sequence Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVQFNWYVDGVEVHNAKTKPREEQFNSTFRVVSVLTVVHQDWLNGKEYKCKVSNKALPAPEEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 10 - protein, 107 aa, Artificial Sequence [sequence omitted]

SEQ ID NO: 11 - protein, 451 aa, Artificial Sequence
  source: /note=“Description of Artificial Sequence Synthetic polypeptide”
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVQFNWYVDGVEVHNAKTKPREEQFNSTFRVVSVLTVVHQDWLNGKEYKCKVSNKALPAPEEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 12 - protein, 219 aa, Artificial Sequence
  source: /note=“Description of Artificial Sequence Synthetic polypeptide”
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 556 aa, Homo sapiens [sequence omitted]

SEQ ID NO: 14 - protein, 5 aa, Artificial Sequence
  source: /note=“Description of Artificial Sequence Synthetic peptide”
GYGMS

SEQ ID NO: 15 - protein, 17 aa, Artificial Sequence
  source: /note=“Description of Artificial Sequence Synthetic peptide”
TITSGGTYTYYPDSVKG

SEQ ID NO: 16 - protein, 9 aa, Artificial Sequence
  source: /note=“Description of Artificial Sequence Synthetic peptide”
SLAGNAMDY

SEQ ID NO: 17 - protein, 8 aa, Artificial Sequence
  source: /note=“Description of Artificial Sequence Synthetic peptide”
RASQTISD

SEQ ID NO: 18 - protein, 7 aa, Artificial Sequence
  source: /note=“Description of Artificial Sequence Synthetic peptide”
FASQSIS

SEQ ID NO: 19 - protein, 9 aa, Artificial Sequence
  source: /note=“Description of Artificial Sequence Synthetic peptide”
QNGHGFPRT

SEQ ID NO: 20 - protein, 118 aa, Artificial Sequence
  source: /note=“Description of Artificial Sequence Synthetic polypeptide”
EVQLVESGGDLVKPGGSLKLSCAASGFTFSGYGMSWVRQTPDKRLEWVATITSGGTYTYYPDSVKGRFTISRDNAKNTLYLQIDSLKSEDTAIYFCARSLAGNAMDYWGQGTSVTVSS

SEQ ID NO: 21 - protein, 107 aa, Artificial Sequence
  source: /note=“Description of Artificial Sequence Synthetic polypeptide”
DIVMTQSPATLSVTPGDRVSLSCRASQTISDYLHWYQQKSHESPRLLIKFASQSISGIPSRFSGSGSGSDFTLSINSVEPEDVGVYYCQNGHGFPRTFGGGTKLEIK

SEQ ID NO: 22 - protein, 5 aa, Artificial Sequence
  source: /note=“Description of Artificial Sequence Synthetic peptide”
NYNMH

SEQ ID NO: 23 - protein, 17 aa, Artificial Sequence
  source: /note=“Description of Artificial Sequence Synthetic peptide”
TIYPGNDDTSYNQKFKD

SEQ ID NO: 24 - protein, 8 aa, Artificial Sequence
  source: /note=“Description of Artificial Sequence Synthetic peptide”
GGYRAMDY

SEQ ID NO: 25 - protein, 16 aa, Artificial Sequence
  source: /note=“Description of Artificial Sequence Synthetic peptide”
RSSQSIVYSNGNTYLG

SEQ ID NO: 26 - protein, 7 aa, Artificial Sequence
  source: /note=“Description of Artificial Sequence Synthetic peptide”
KVSNRFS

SEQ ID NO: 27 - protein, 9 aa, Artificial Sequence
  source: /note=“Description of Artificial Sequence Synthetic peptide”
FQGSHVPYT

SEQ ID NO: 28 - protein, 117 aa, Artificial Sequence
  source: /note=“Description of Artificial Sequence Synthetic polypeptide”
QVQLVQSGAEVKKPGASVKVSCKASGYTFTNYNMHWVRQAPGQGLEWIGTIYPGNDDTSYNQKFKDKATLTADKSTSTAYMELSSLRSEDTAVYYCARGGYRAMDYWGQGTLVTVSS

SEQ ID NO: 29 - protein, 112 aa, Artificial Sequence
  source: /note=“Description of Artificial Sequence Synthetic polypeptide”
DVVMTQSPLSLPVTPGEPASISCRSSQSIVYSNGNTYLGWYLQKPGQSPKLLIYKVSNRFSGVPDRFSGSGSGTDFTLKISRVEAEDVGVYHCFQGSHVPYTFGGGTKVEIK

SEQ ID NO: 30 - protein, 117 aa, Artificial Sequence
  source: /note=“Description of Artificial Sequence Synthetic polypeptide”
QVQLVQSGAEVKKPGASVKVSCKASGYTFTNYNMHWVRQAPGQRLEWMGTIYPGNDDTSYNQKFKDRVTITADTSASTAYMELSSLRSEDTAVYYCARGGYRAMDYWGQGTLVTVSS

SEQ ID NO: 31 - protein, 112 aa, Artificial Sequence
  source: /note=“Description of Artificial Sequence Synthetic polypeptide”
DIVMTQSPLSLPVTPGEPASISCRSSQSIVYSNGNTYLGWYLQKPGQSPQLLIYKVSNRFSGVPDRFSGSGSGTDFTLKISRVEAEDVGVYYCFQGSHVPYTFGQGTKLEIK

SEQ ID NO: 32 - protein, 117 aa, Artificial Sequence
  source: /note=“Description of Artificial Sequence Synthetic polypeptide”
QVQLVQSGAEVKKPGASVKVSCKASGYTFTNYNMHWVRQAPGQRLEWIGTIYPGNDDTSYNQKFKDRATLTADKSASTAYMELSSLRSEDTAVYYCARGGYRAMDYWGQGTLVTVSS

SEQ ID NO: 33 - protein, 112 aa, Artificial Sequence
  source: /note=“Description of Artificial Sequence Synthetic polypeptide”
DVVMTQSPLSLPVTPGEPASISCRSSQSIVYSNGNTYLGWYLQKPGQSPQLLIYKVSNRFSGVPDRFSGSGSGTDFTLKISRVEAEDVGVYHCFQGSHVPYTFGQGTKLEIK

SEQ ID NO: 34 - protein, 444 aa, Artificial Sequence
  source: /note=“Description of Artificial Sequence Synthetic polypeptide”
QVQLVQSGAEVKKPGASVKVSCKASGYTFTNYNMHWVRQAPGQRLEWMGTIYPGNDDTSYNQKFKDRVTITADTSASTAYMELSSLRSEDTAVYYCARGGYRAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 35 - protein, 219 aa, Artificial Sequence
  source: /note=“Description of Artificial Sequence Synthetic polypeptide”
DIVMTQSPLSLPVTPGEPASISCRSSQSIVYSNGNTYLGWYLQKPGQSPQLLIYKVSNRFSGVPDRFSGSGSGTDFTLKISRVEAEDVGVYYCFQGSHVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC